SEQUENCE LISTING (14 sequences)

SEQ ID NO: 1 - DNA, 2633 bp, synthetic construct
  source 1..2633: mol_type = other DNA
ctagcgatgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctggcctgtacggaagtgttacttctgctctaaaagctgcggaattgtacccgcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaattg

SEQ ID NO: 2 - DNA, 1676 bp, synthetic construct
  source 1..1676: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 3 - DNA, 657 bp, synthetic construct
  source 1..657: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaa

SEQ ID NO: 4 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 5 - DNA, 2575 bp, synthetic construct
  source 1..2575: mol_type = other DNA
gagctcgagctagcgatgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctggcctgtacggaagtgttacttctgctctaaaagctgcggaattgtacccgcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaaattgcaattgcat

SEQ ID NO: 6 - DNA, 132 bp, synthetic construct
  source 1..132: mol_type = other DNA
ccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 7 - DNA, 2216 bp, synthetic construct
  source 1..2216: mol_type = other DNA
gaggatatcgataattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacaggcctgtacggaagtgttacttctgctctaaaagctgcggaattgtacccgcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaattgcgatatccattcatttta

SEQ ID NO: 8 - DNA, 509 bp, synthetic construct
  source 1..509: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 9 - DNA, 572 bp, synthetic construct
  source 1..572: mol_type = other DNA
agagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacag

SEQ ID NO: 10 - DNA, 1778 bp, synthetic construct
  source 1..1778: mol_type = other DNA
cagctagcgatcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaattgcaattgcattctatgtttcaggt

SEQ ID NO: 11 - DNA, 1696 bp, synthetic construct
  source 1..1696: mol_type = other DNA
gagctagcgatcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaaattgcaattgcattcatttta

SEQ ID NO: 12 - DNA, 1744 bp, synthetic construct
  source 1..1744: mol_type = other DNA
cagctagcgataattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaattgcaattgcattctatgtttcaggt

SEQ ID NO: 13 - DNA, 1524 bp, synthetic construct
  source 1..1524: mol_type = other DNA
gagctagcgataattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggagttcagatcttattaaggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaattgcaattgcattcatttta

SEQ ID NO: 14 - DNA, 2108 bp, synthetic construct
  source 1..2108: mol_type = other DNA
tacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgtttagtgaaccgtcagatcgcctggagacgccatccacgctgttttgacctccatagaagacaccgggaccgatccagcctccgcggccgggaacggtgcattggaagcttgccaccatgaaggccatcctggtggtgctgctgtacaccttcgccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagagcctgagcaccgccagcagctggagctacatcgtggagaccagcagcagcgacaacggcacctgctaccccggcgacttcatcgactacgaggagctgcgggagcagctgagcagcgtgagcagcttcgagcggttcgagatcttccccaagaccagcagctggcccaaccacgacagcaacaagggcgtgaccgccgcctgcccccacgccggcgccaagagcttctacaagaacctgatctggctggtgaagaagggcaacagctaccccaagctgagcaagagctacatcaacgacaagggcaaggaggtgctggtgctgtggggcatccaccaccccagcaccagcgccgaccagcagagcctgtaccagaacgccgacgcctacgtgttcgtgggcaccagccggtacagcaagaagttcaagcccgagatcgccatccggcccaaggtgcgggaccaggagggccggatgaactactactggaccctggtggagcccggcgacaagatcaccttcgaggccaccggcaacctggtggtgccccggtacgccttcgccatggagcggaacgccggcagcggcatcatcatcagcgacacccccgtgcacgactgcaacaccacctgccagacccccaagggcgccatcaacaccagcctgcccttccagaacatccaccccatcaccatcggcaagtgccccaagtacgtgaagagcaccaagctgcggctggccaccggcctgcggaacgtgcccagcatccagagccggggcctgttcggcgccatcgccggcttcatcgagggcggctggaccggcatggtggacggctggtacggctaccaccaccagaacgagcagggcagcggctacgccgccgacctgaagagcacccagaacgccatcgacgagatcaccaacaaggtgaacagcgtgatcgagaagatgaacacccagttcaccgccgtgggcaaggagttcaaccacctggagaagcggatcgagaacctgaacaagaaggtggacgacggcttcctggacatctggacctacaacgccgagctgctggtgctgctggagaacgagcggaccctggactaccacgacagcaacgtgaagaacctgtacgagaaggtgcggagccagctgaagaacaacgccaaggagatcggcaacggctgcttcgagttctaccacaagtgcgacaacacctgcatggagagcgtgaagaacggcacctacgactaccccaagtacagcgaggaggccaagctgaaccgggaggagatcgacggcgtgaagctggagagcacccggatctaccagatcctggccatctacagcaccgtggccagcagcctggtgctggtggtgagcctgggcgccatcagcttctggatgtgcagcaacggcagcctgcagtgccggatctgcatctgatagtctaga

(19) United States
(12) Patent Application Publication
(10) Pub. No.: US 2026/0201417 A1
(43) Pub. Date: Jul. 16, 2026

(54) TET-R EXPRESSION CASSETTES AND CELL LINES

(71) Applicant: CEVEC PHARMACEUTICALS GMBH, Cologne (DE)

(72) Inventors: M. Scot Roberts, Gaithersburg, MD (US); Jianfeng Zhang, Gaithersburg, MD (US)

(21) Appl. No.: 19/137,596

(22) PCT Filed: Dec. 11, 2023
(86) PCT No.: PCT/EP2023/085187

§ 371 Date: Jun. 10, 2025

Related U.S. Application Data
(60) Provisional application No. 63/431,717, filed on Dec. 11, 2022.

(51) Int. Cl. C12N 15/861; C07K 14/11; C07K 14/165; C12N 5/073; C12N 7/02; C12N 7/04; C12N 15/11

(52) U.S. Cl. CPC C12N 15/861; C07K 14/11; C07K 14/165; C12N 5/0605; C12N 7/025; C12N 7/045; C12N 15/11; C12N 2510/04; C12N 2710/10023; C12N 2710/10042; C12N 2710/10043; C12N 2710/10044; C12N 2710/10052; C12N 2830/006; C12N 2830/34; C12N 2830/50

(57) ABSTRACT

This disclosure relates to a cell line containing polynucleotides comprising at least one Tetracycline operon repressor protein (TetR) expression cassette for producing recombinant Adenoviral (Ad) vectors encoding otherwise toxic transgenes.

### FIELD OF THE DISCLOSURE

This disclosure relates to an adenovirus early region 1 (E1)-complementing cell line containing integrated polynucleotides comprising at least one Tetracycline operon repressor protein (TetR) expression cassette for to allow the repression of E1-deleted adenoviral vectors encoding otherwise toxic transgenes under a promotor containing one or more tetracycline operators (TetO).

### BACKGROUND OF THE DISCLOSURE

An art-recognized problem is that there are manufacturing limitations of non-replicating E1 and/or E3 deletion Adenoviral vectors (“Ad vector”) containing certain toxic protein-encoding transgenes (as used herein “toxic” refers to a protein-encoding transgene that when expressed interferes with propagation and/or packaging of viral particles during production using a E1-complementing cell line), such as but not limited to pro-apoptotic procaspase 8 protein-coding gene, B-cell lymphoma 2 (BCL2) associated X, Fas cell surface death receptor ligand protein-coding gene, Envelope protein-encoding gene from hepatitis C, Envelope protein-coding gene from HIV, circumsporozoite (CS) protein-encoding gene from plasmodium and EBO protein-encoding gene from Ebola virus, certain influenza hemagglutinin (HA) protein-encoding gene, spike protein-coding gene from severe acute respiratory syndrome coronavirus (SARS-CoV1/2) or other human or virus protein-encoding genes. This problem cannot be resolved with standard procedures, which result in very low yield. The reagents and methods disclosed herein provide solutions to such art-recognized problems.

### BRIEF DESCRIPTION OF THE DRAWINGS

FIG. 1: Sequence of TetR expression cassette #1 (SEQ ID NO: 1). CAG/Chimeric intron sequence is indicated with single underline; TetR gene sequence is in bold character and a BGH polyA sequence is indicated with double underline.

FIG. 2: Sequence of CAG promoter/Chimeric intron (SEQ ID NO: 2)

FIG. 3: Sequence of the TetR gene (SEQ ID NO: 3)

FIG. 4: Sequence of BGH polyA (SEQ ID NO: 4)

FIG. 5: Sequence of TetR expression cassette #2 (SEQ ID NO: 5). CAG/Chimeric intron sequence is indicated with single underlined; TetR gene sequence is in bold character and the SV40 polyA sequence is indicated with double underlined.

FIG. 6: Sequence of SV40 polyA (SEQ ID NO: 6)

FIG. 7: Sequence of TetR expression cassette #3 (SEQ ID NO: 7). CMV promoter sequence is indicated with single underlined; The β-Globin intron sequence is indicated in lower case; TetR gene sequence is in bold character and the BGH polyA sequence is indicated with double underlined.

FIG. 8: Sequence of CMV promoter (SEQ ID NO: 8)

FIG. 9: Sequence of β-Globin intron (SEQ ID NO: 9)

FIG. 10: Sequence of TetR expression cassette #4 (SEQ ID NO: 10).

FIG. 11: Sequence of TetR expression cassette #5 (SEQ ID NO: 11).

FIG. 12: Sequence of TetR expression cassette #6 (SEQ ID NO: 12).

FIG. 13: Sequence of TetR expression cassette #7 (SEQ ID NO: 13).

FIG. 14: Detection of TetR in CAP-TetR Cells Z3634 and Z3635 by western blot

FIG. 15: Detection of TetR in CAP-TetR Cells C235 and C236 by western blot

FIG. 16: Detection of TetR in CAP-TetR Cells Z3616, Z3617 and Z3618 by western blot

FIG. 17: Synthesized 2×TetO-coCA09 DNA fragment (SEQ ID NO: 14). The double TetO sequence is in bold and italics and the coCA09 HA sequence (codon optimized HA gene of influenza A(H1N1) pdm09 virus) is underlined.

### DETAILED DESCRIPTION OF THE DISCLOSURE

As mentioned above, art-recognized problems that the subject matter of this disclosure solves relate to the manufacturing limitations encountered with non-replicating E1 and/or E3 deletion Adenoviral vectors (“Ad vector”) expressing certain toxic transgenes. As used herein, “toxic” refers to a transgene that when expressed produces a polypeptide (a “toxic polypeptide”) that interferes with propagation and/or packaging of viral particles during production using a E1 complementing cell line. In embodiments, the transgenes are inserted in the E1 location of the Ad vector. In some embodiments, the toxic polypeptide can be derived from an organism selected from the group consisting of Actinomyces, Anabaena, Bacillus (e.g., Bacillus anthracis such as protective antigen, lethal factor, or edema factor), Bacteroides, Bordetella (e.g., Bordetella pertussis such as adenylate cyclase toxin or pertussis toxin), Caulobacter, Chlamydia, Chlorobium, Chromatium, Clostridium (e.g., Clostridium perfringens such as perfringens enterotoxin), Clostridium botulinum such as botulinum toxin), or Clostridium tetani such as tetanus toxin)), Corynebacterium diphtheriae (such as diphtheria toxin), Cytophaga, Deinococcus, Escherichia (e.g., Escherichia coli such as ST toxin or LT toxin), Halobacterium, Heliobacter, Hyphomicrobium, Methanobacterium, Micrococcus, Myobacterium, Mycoplasma, Myxococcus, Neisseria, Nitrobacter, Oscillatoria, Prochloron, Proteus, Pseudomonas (e.g., Pseudomonas aeruginosa such as exotoxin A), Phodospirillum, Rickettsia, Salmonella, Shigella (e.g., Shigella dysenteriae such as shiga toxin), Spirillum, Spirochaeta, Staphylococcus (e.g., Staphylococcus aureus such as enterotoxin, toxic shock syndrome toxin, or exfoliatin toxin), Streptococcus (e.g., Streptococcus pyogenes such as erythrogenic toxin), Streptomyces, Sulfolobus, Thermoplasma, Thiobacillus, Treponema, Vibrio cholerae (such as cholera enterotoxin), Sporozoa (e.g., Plasmodium), Ciliophora, rhizopoda, Zoomastigophora. Preferably, the parasite is of the phylum Sporozoa and genus Plasmodium (e.g., circumsporozoite protein (CSP), sporozoite surface protein 2 (SSP2), liver-stage antigen 1 (LSA-1), Pf exported protein 1 (PfExp-1)/Py hepatocyte erythrocyte protein 17 (PyHEP17), Pf Antigen 2, merozoite surface protein 1 (MSP-1), merozoite surface protein 2 (MSP-2), erythrocyte binding antigen 175 (EBA-175), ring-infected erythrocyte surface antigen (RESA), serine repeat antigen (SERA), glycophorin binding protein (GBP-130), histidine rich protein 2 (HRP-2), rhoptry-associated proteins 1 and 2 (RAP-1 and RAP-2), erythrocyte membrane protein 1 (PfEMP1), or apical membrane antigen 1 (AMA-1)), Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae (e.g., Hepatitis B virus), Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia virus), Reoviridae (e.g., rotavirus), Retroviridae (e.g., lentivirus, such as human immunodeficiency virus (HIV) 1 and HIV 2), Rhabdoviridae, Totiviridae, Crimean-Congo haemorrhagic fever virus, Eastern Equine Encephalitis virus, Hendra virus, Lassa fever virus, Monkeypox virus, Nipah virus, Rift Valley fever virus, South American Haemorrhagic Fever viruses, Venezuelan Equine Encephalitis virus, Human Immunodeficiency Virus (HIV, such as the gag, env, or pol proteins), foot-and-mouth disease virus (FMDV) protein, Dengue, Coccidioides, Candida, Cryptococcus, Trichosporon, Acremonium, Cladophialophora, Pseudallescheria, Rizopus, Scedosporium, Aspergillus, Aureobasidium, Bipolaris, Fusarium, Phialophora, Blastomyces, Histoplasma, Sporothrix, a plant (e.g., lectins such as ricin or abrin), alkaloids, glycosides, oxalates, phenols, resins, volatile oils, and phototoxins such as coumarins), or animal (e.g., transforming growth factor β (TGFβ), or nitric oxide synthase (NOS)). In embodiments, the transgene encodes at least one of pro-apoptotic procaspase 8, BCL2 associated X, Fas cell surface death receptor ligand, Envelope proteins from hepatitis C, Envelope proteins from HIV, circumsporozoite (CS) protein from plasmodium and EBO from Ebola virus, certain influenza hemagglutinin (HA) gene, spike protein from SARS-CoV1/2 and/or other antigens that could not be rescued or produced with sufficient yield using standard procedures.

This disclosure provides reagents and methods of propagating an Ad vector comprising a transgene encoding a polypeptide that would otherwise be toxic to the Ad host cell. The reagents and methods disclosed herein provide cells expressing a tetracycline operon repressor protein (TetR) and infecting the cells with an Ad vector comprising a heterologous transgene encoding a toxic polypeptide and at least one tetracycline operon operator sequences (TetO). In preferred embodiments, the Ad vector polynucleotide encoding the transgene is operably linked to a promoter and at least one or more TetO sequences (TetO). Expression of the toxic polypeptide in the host cells is inhibited in the presence of TetR such that the Ad vector replicates in the cell. TetR inhibits expression of the transgene when bound to the TetO operon sequence(s).

In some embodiments, this disclosure provides Tetracycline Repressor protein (TetR) expression cassettes and transfected cell lines thereof, wherein the TetR expression cassettes are preferably integrated into the genome of the cell. Preferred TetR expression cassettes comprise polynucleotides including at least one promoter, at least one intron, at least one TetR coding sequence, and at least one poly(A) sequence. In preferred embodiments, the cells in which the Ad vector is propagated comprises at least one of such TetR expression cassettes. In some embodiments, the TetR polypeptide encoded by the TetR expression cassette(s) can having the amino acid sequence shown below (presented as standard one letter amino acid symbols): MSRLDKSKVINSALELLNEVGIEGLTTRKLAQKLGVEQPTLYWHVKNKRALLDALAIEMLDRHH THFCPLEGESWQDELRNNAKSFRCALLSHRDGAKVHLGTRPTEKQYETLENOLAFLCQQGFSLE NALYALSAVGHFTLGCVLEDQEHQVAKEERETPTTDSMPPLLRQAIELFDHOGAEPAFLFGLEL IICGLEKQLKCESGSAYSGSREFRSY (SEQ ID NO: 14); or a suitable derivative thereof (e.g., GenBank Accession No. J01830; conservative substitution of positively-charged residues (H, K, and R) substituted by other positively charged residues, negatively-charged residues (D and E) are substituted with other negatively-charged residues, neutral polar residues (C, G, N, Q, S, T, and Y) are substituted with other neutral polar residues, and neutral non-polar residues (A, F, I, L, M, P, V, and W) are substituted with other neutral non-polar residues). In some embodiments, the TetR protein can be encoded by the polynucleotide sequence shown below: ATGTCTAGATTAGATAAAAGTAAAGTGATTAACAGCGCATTAGAGCTGCTTAATGAGGTCGGAA TCGAAGGTTTAACAACCCGTAAACTCGCCCAGAAGCTAGGTGTAGAGCAGCCTACATTGTATTG GCATGTAAAAAATAAGCGGGCTTTGCTCGACGCCTTAGCCATTGAGATGTTAGATAGGCACCAT ACTCACTTTTGCCCTTTAGAAGGGGAAAGCTGGCAAGATTTTTTACGTAATAACGCTAAAAGTT TTAGATGTGCTTTACTAAGTCATCGCGATGGAGCAAAAGTACATTTAGGTACACGGCCTACAGA AAAACAGTATGAAACTCTCGAAAATCAATTAGCCTTTTTATGCCAACAAGGTTTTTCACTAGAG AATGCATTATATGCACTCAGCGCTGTGGGGCATTTTACTTTAGGTTGCGTATTGGAAGATCAAG AGCATCAAGTCGCTAAAGAAGAAAGGGAAACACCTACTACTGATAGTATGCCGCCATTATTACG ACAAGCTATCGAATTATTTGATCACCAAGGTGCAGAGCCAGCCTTCTTATTCGGCCTTGAATTG ATCATATGCGGATTAGAAAAACAACTTAAATGTGAAAGTGGGTCCGCGTACAGCGGATCCCGGG AATTCAGATCTTATTAA (SEQ ID NO: 3); or a polynucleotide encoding the TetR polypeptide but having at least about 90% identity thereto that maintains the function of TetR (e.g., a conservatively substituted derivative). Codons that can be used to encode such nearly identical TetR polypeptides are well understood by those of ordinary skill in the art.

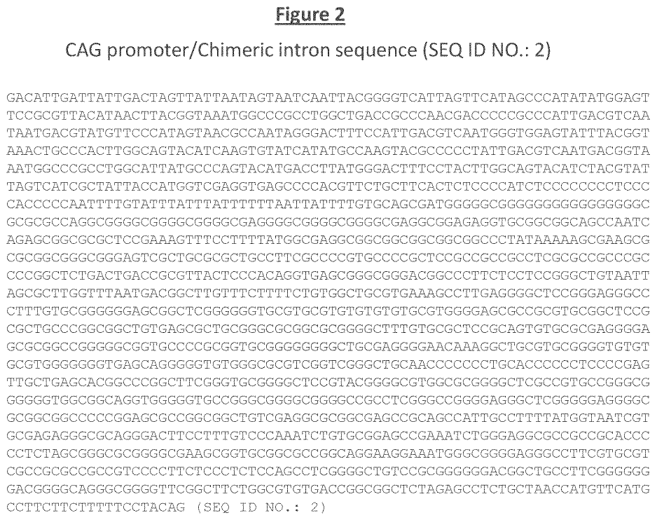

Any suitable promoters can be used with the other elements disclosed herein as may be determined by those of ordinary skill in the art. For instance, a suitable promoter can include a cytomegalovirus (CMV) promoter, such as the CMV immediate-early promoter (described in, for example, U.S. Pat. Nos. 5,168,062 and 5,385,839, and GenBank accession number X17403) or SEQ ID NO: 8 herein (FIG. 8); an HIV promoter (e.g., the HIV long terminal repeat promoter); Rous sarcoma virus (RSV) promoters (e.g., RSV long terminal repeat); mouse mammary tumor virus (MMTV) promoter; HSV promoter (e.g., Lap2 promoter or the herpes thymidine kinase promoter (Wagner et al., Proc. Natl. Acad. Sci., 78, 144-145 (1981)); an SV40 promoter; and Epstein Barr virus promoter; an adeno-associated viral promoter (e.g., the p5 promoter); and/or the like. In preferred embodiments, the promoter is the CAG promoter illustrated in FIG. 2 (within SEQ ID NO: 2).

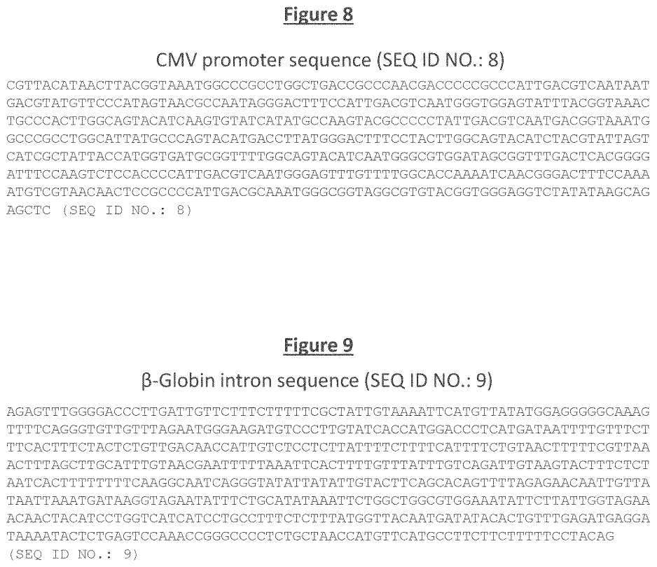

Any suitable intron sequence may also be used as may be determined by those of ordinary skill in the art. In preferred embodiments, the intron sequence is the β-globin intron sequence shown in FIG. 9 (SEQ ID NO: 9). In some preferred embodiments, the intron sequence is a chimeric intron sequences illustrated in FIG. 2 (within SEQ ID NO: 2).

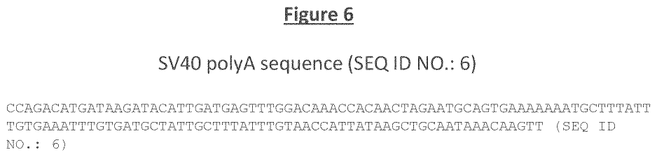

Any suitable poly(A) polynucleotide sequence may also be used as may be determined by those of ordinary skill in the art. In some preferred embodiments, the poly(A) sequence is the bovine growth hormone (BGH) polynucleotide sequence shown in FIG. 4 (SEQ ID NO: 4). In some preferred embodiments, the poly(A) sequence is the SV40 polynucleotide sequence shown in FIG. 6 (SEQ ID NO: 6).

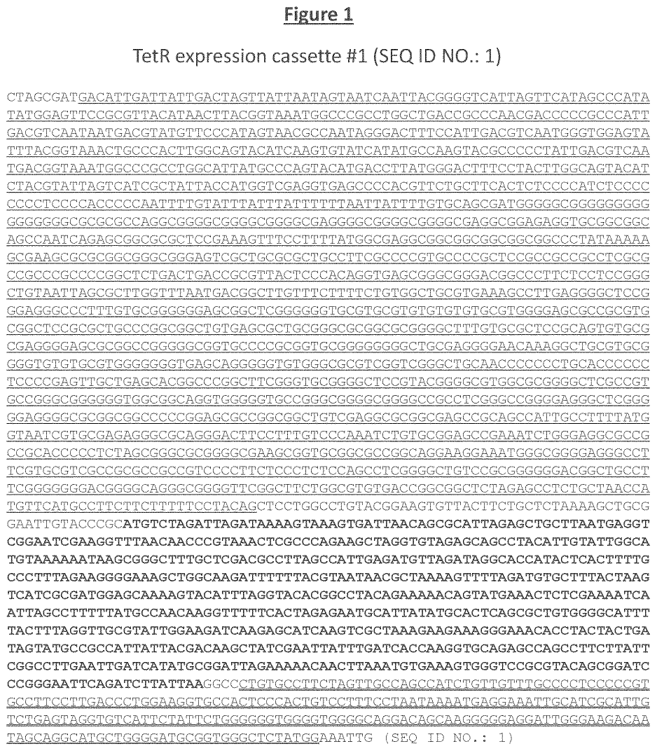

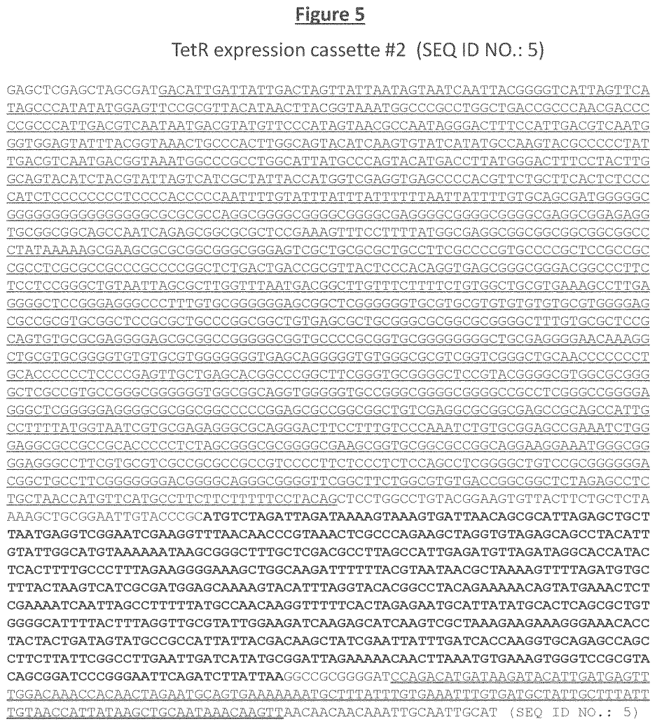

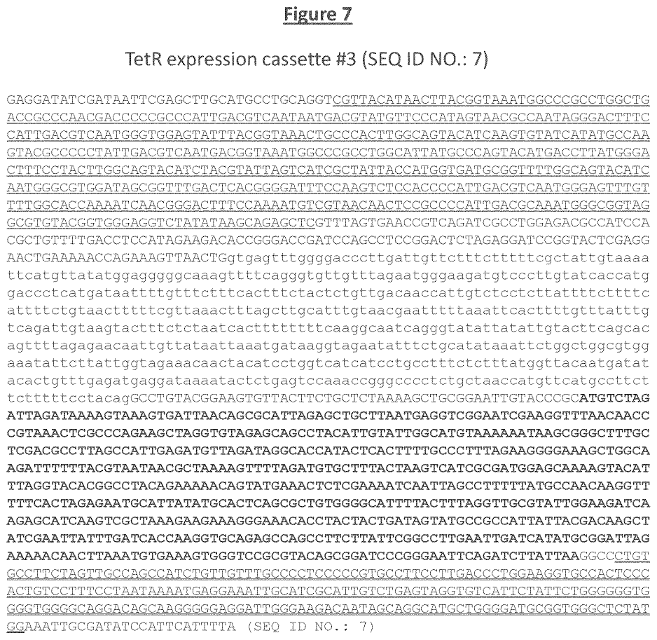

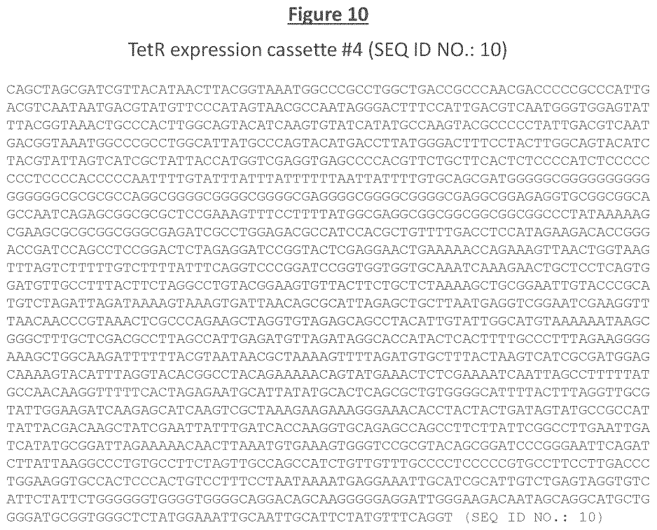

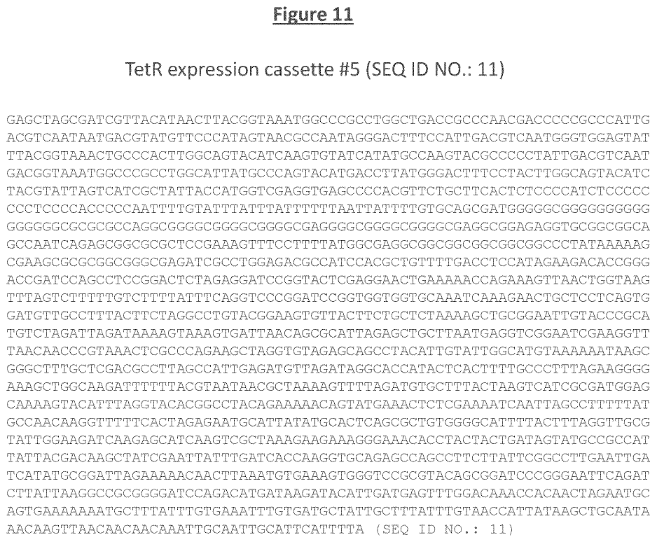

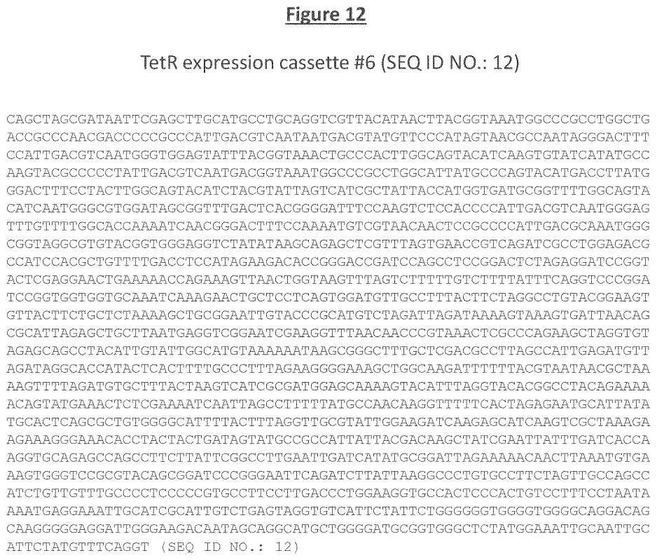

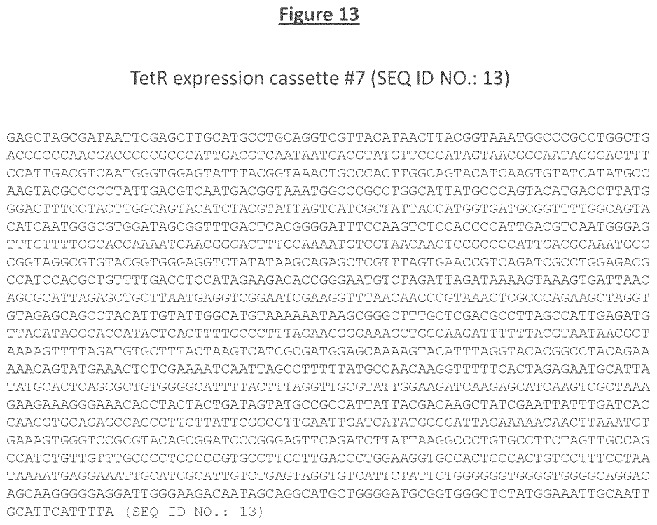

As mentioned above, in certain embodiments, the cells in which the Ad vector is propagated comprise at least one of the TetR expression cassettes disclosed herein. Other suitable TetR expression cassettes may also be suitable as may be determined by those of ordinary skill in the art. In preferred embodiments, the TetR expression cassette is that illustrated in FIG. 1 (SEQ ID NO: 1; TetR expression cassette #1), FIG. 5 (SEQ ID NO: 5; TetR expression cassette #2), FIG. 7 (SEQ ID NO. 7; TetR expression cassette #3), FIG. 10 (SEQ ID NO. 10; TetR expression cassette #4), FIG. 11 (SEQ ID NO. 11; TetR expression cassette #5), FIG. 12 (SEQ ID NO. 12; TetR expression cassette #6), or FIG. 13 (SEQ ID NO. 13; TetR expression cassette #7); or a TetR expression cassette at least about 90% identical thereto and is able to carry out the functions disclosed herein.

The TetO polynucleotide sequence (e.g., TetO site) is comprised within the Ad vector and can be any suitable TetO polynucleotide sequence that inhibits expression of the polynucleotide encoding the toxic polypeptide in the presence of TetR in the Ad vector host cell. In a preferred embodiment, the polynucleotide encoding the toxic polypeptide is operably linked to one or more TetO sites. In some embodiments, the TetO site can be and/or comprise the polynucleotide sequence: AGCTCTCCCTATCAGTGATAGAGATCTCCCTATCAGTGATAGAGATCGTCGACGAGCT (SEQ ID NO: 15); or any derivative thereof that maintains the TetO function described above.

In preferred embodiments, the TetO polynucleotide sequence is:

(SEQ ID NO: 16)
TCCCTATCAGTGATAGAGATCTCCCTATCAGTGATAGAGATCGTCGAC.

The polynucleotide encoding the toxic polypeptide (i.e., the transgene of the Ad vector) is operably linked to at least one, but preferably at least two, such TetO polynucleotide sequences. The TetO polynucleotide sequence(s) can be positioned upstream (e.g., 5′), between, or downstream (e.g., 3′) of the promoter and/or the polynucleotide sequence encoding the toxic polypeptide.

In some embodiments, the cell line is an immortalized, E1-complementing human amniocyte-derived cell line (herein referred to as “CAP-TetR” cells) developed to rescue and produce non-replicating E1/E3 deletion adenoviral vectors encoding toxic transgenes which are difficult to generate and amplify using standard procedures. The CAP TetR cells express TetR which inhibits expression of the toxic polypeptide encoded by the Ad vector polynucleotide encoding the transgene, the expression of which is controlled by a promoter (preferably the CMV promoter) and (or comprising) a TetO operon sequence (SEQ ID NO: 16) to which TetR binds.

In some embodiments, this disclosure provides CAP-TetR cells that consistently express TetR protein, wherein the cells comprise an Ad vector comprising one or more, preferably two, copies of Tetracycline operator (TetO) sequence (Ad2×tetO-Vector which includes two copies of the TetO operon) in the cytomegalovirus (CMV) promoter. Without being limited by the mode of operation, it is believed that in such CAP-TetR cells, the TetR protein binds to the TetO sequence of Ad2×TetO-Vectors, blocking toxic transgene expression, and allowing Ad2×TetO-Vectors replication and propagation of to be rescued efficiently. These Ad2×TetO-Vectors could express transgene in high levels after infection of host cells for vaccination and gene therapy.

As shown in the Examples, in one preferred embodiment, the CAP-TetR cells were obtained using recombinant DNA technologies provided by CEVEC Pharmaceuticals GmbH (Germany; “CEVEC”) Amniocyte Production (CAP) cells, an immortalized cell line based on primary human amniocytes. CAP cells are of non-tumor origin, are immortalized by a function not oncogenic in human, and are obtained from an ethically accepted source of origin. Primary human amniocytes were obtained by routine amniocentesis have been immortalized with a vector containing the functions E1 and pIX of adenovirus type 5. Consequently, CAP cell lines permanently express the E1 protein of adenovirus type 5 complementing Ad vectors allowing replication during production. CAP cells have been developed to avoid the emergence of Replication-Competent Adenovirus (RCA) contamination as observed with other E1-complementing cell lines such as HEK293 cells. CAP cells grow as single cell suspension in chemically defined serum-free medium suitable for the production of pharmaceutical products.

In some preferred embodiments, this disclosure provides recombinant polynucleotide comprising at least one Tetracycline operon repressor protein (TetR) expression cassette (TetR expression cassette), wherein each TetR expression cassette comprises: a CAG promoter/chimeric intron sequence that is optionally SEQ ID NO: 2; and, a poly(A) polynucleotide sequence that is optionally SEQ ID NO: 4 or SEQ ID NO: 6, operably linked to a coding sequence for the TetR coding sequence that is optionally SEQ ID NO: 3; optionally wherein the recombinant polynucleotide comprising a sequence having at least about 90% identity with SEQ ID NOs. 2, 3, 4 and/or 6. In some preferred embodiments, the recombinant polynucleotide of claim 1 wherein said TetR expression cassette is SEQ ID NO.: 1 or SEQ ID NO.: 5, or an expression cassette having at least about 90% identity therewith. In some preferred embodiments, this disclosure provides a recombinant polynucleotide comprising at least one Tetracycline operon repressor protein (TetR) expression cassette that is optionally SEQ ID NO: 7 or an expression cassette having at least about 90% identity therewith; wherein each TetR expression cassette comprises a CMV promoter sequence that is optionally SEQ ID NO: 8 or a sequence having at least about 90% identity therewith, a rabbit beta-globin intron sequence that is optionally SEQ ID NO: 9 or a sequence having at least about 90% identity therewith, and a poly(A) polynucleotide sequence operably linked to a coding sequence for the TetR expression sequence is optionally SEQ ID NO: 3 or a sequence having at least about 90% identity therewith. In some preferred embodiments, this disclosure provides one or more TetR expressing cells comprising a recombinant polynucleotide disclosed herein integrated into the CAP cell genome. In some preferred embodiments, the TetR expressing cell comprises a TetR expression cassette selected from the group consisting of SEQ ID NO: 1, SEQ ID NO: 5, SEQ ID NO: 7, and a TetR expression cassette having at least about 90% identity therewith. In some preferred embodiments, the cell is a human cell. In some preferred embodiments, the human cell is an immortalized human amniocyte cell line such as a CAP cell.

In some preferred embodiments, this disclosure provides methods for producing an adenoviral vector encoding a transgene, the method comprising: a) obtaining a Tetracycline operon repressor protein (TetR) expressing cell comprising an exogenous polynucleotide in its genome, the polynucleotide comprising at least one Tetracycline operon repressor protein (TetR) expression cassette, each TetR expression cassette comprising a CAG promoter/chimeric intron sequence that is optionally SEQ ID NO: 2, and a poly(A) polynucleotide sequence that is optionally SEQ ID NO: 4 or SEQ ID NO: 6, operably linked to a coding sequence for the TetR gene that is optionally SEQ ID NO: 3; b) transfecting the TetR expressing cells with at least one Tetracycline operon operator (TetO) polynucleotide sequence linked to the promoter in a recombinant Ad genomic plasmid to produce a replication-deficient Ad vector containing at least one TetO polynucleotide sequence and encoding at least one transgene; and, c) isolating the Ad vector particles containing at least one TetO polynucleotide sequence and encoding at least one transgene; wherein: the polynucleotides of part a) have at least about 90% identity with any of SEQ ID NOs. 2, 3, 4 or 6; and, the number of Ad vector particles containing at least one TetO polynucleotide sequence and encoding at least one transgene isolated in step c) is at least about any of two, 10, 15, 20, 50, 100, 150, 200, 250, or 300 times the number of Ad vector particles obtained from an Ad vector package cell lacking the TetR polynucleotide. In some preferred embodiments, the TetR expression cassette comprising SEQ ID NO: 1 or SEQ ID NO: 5, or an expression cassette having at least about 90% identity therewith. In some preferred embodiments, the chimeric intron comprises a chicken beta actin intron sequence and a rabbit beta goblin intron sequence. In some preferred embodiments, the CAG/chimeric intron sequence is according to SEQ ID NO: 2, or an intron sequence having at least about 90% identity therewith. In some preferred embodiments, the CAG/chimeric intron comprises SEQ ID NO: 2 or sequence having at least about 90% identity therewith, the poly(A) polynucleotide sequence is BGH (SEQ ID NO: 4) or a sequence having at least about 90% identity therewith, the transgene comprises a coronavirus spike protein, and the number of Ad vector particles is at least about any of 20, 60 or 200 times the number of Ad vector particles obtained from an Ad vector package cell lacking the TetR polynucleotide. In some preferred embodiments, the CAG/chimeric intron comprises SEQ ID NO: 2 or sequence having at least about 90% identity therewith, the poly(A) polynucleotide sequence is SV40 (SEQ ID NO: 6) or sequence having at least about 90% identity therewith, the transgene comprises a coronavirus spike protein, and the number of Ad vector particles is at least about any of 30, 150 or 200 times the number of Ad vector particles obtained from an Ad vector package cell lacking the TetR polynucleotide. In some preferred embodiments, the CAG/chimeric intron comprises SEQ ID NO: 2 or sequence having at least about 90% identity therewith, the poly(A) polynucleotide sequence is a BGH poly(A) polynucleotide sequence (SEQ ID NO: 4) or sequence having at least about 90% identity therewith, the transgene comprises an influenza hemagglutinin (HA) surface protein antigen from A/Perth/16/2009(H3N2), and the number of Ad vector particles is at least any of about two, 10, or 20 times the number of Ad vector particles obtained from an Ad vector package cell lacking the TetR polynucleotide. In some preferred embodiments, the CAG/chimeric intron comprises SEQ ID NO: 2 or sequence having at least about 90% identity therewith, the poly(A) polynucleotide sequence is a SV40 poly(A) polynucleotide sequence (SEQ ID NO: 6) or sequence having at least about 90% identity therewith, the transgene comprises an influenza hemagglutinin (HA) surface protein antigen from A/Perth/16/2009(H3N2), and the number of Ad vector particles is at least any of about five or 10 times the number of Ad vector particles obtained from an Ad vector package cell lacking the TetR polynucleotide.

In some preferred embodiments, this disclosure provides methods for producing an adenoviral vector encoding a transgene, the method comprising: a) obtaining a Tetracycline operon repressor protein (TetR) recombinant cell comprising an exogenous polynucleotide in its genome, the polynucleotide comprising at least one Tetracycline operon repressor protein (TetR) expression cassette, each TetR expression cassette comprising a CMV promoter sequence (SEQ ID NO: 8), a rabbit beta globin intron sequence (SEQ ID NO: 9), and a poly(A) polynucleotide sequence (SEQ ID NO: 4) operably linked to a coding sequence for the TetR gene (SEQ ID NO: 3); b) transfecting the TetR expressing cells with at least one Tetracycline operon operator (TetO) polynucleotide sequence linked to the promoter in a recombinant Ad genomic plasmid to a replication-deficient Ad vector containing at least one TetO polynucleotide sequence and encoding at least one transgene; and, c) isolating the Ad vector particles containing at least one TetO polynucleotide sequence and encoding at least one transgene; wherein: the polynucleotides of part a) have at least about 90% identity with any of SEQ ID NOs. 3, 48, or 9; and the number of Ad vector particles containing at least one TetO polynucleotide sequence and encoding at least one transgene isolated in step c) is at least about any of two, 10, 15, 20, 50, 100, 150, 200, 250, or 300 times the number of Ad vector particles obtained from an Ad vector packaging cell (e.g., host cell, cell in which the Ad vector is propagated) lacking the TetR polynucleotide. In some preferred embodiments, the TetR expression cassette is according to SEQ ID NO: 7 or an expression cassette have at least about 90% identity therewith. In some preferred embodiments, the intron sequence is SEQ ID NO: 9 or an intron sequence having at least about 90% identity therewith.

In some preferred embodiments, the poly(A) polynucleotide sequence used in such methods is a BGH or SV40 poly(A) polynucleotide sequence, optionally SEQ ID NO: 4 or SEQ ID NO: 6 or a polynucleotide sequence having at least about 90% identity therewith. In some preferred embodiments, the transgene is a viral or bacterial antigen. In some preferred embodiments, the viral antigen is a coronavirus or flu antigen.

In some preferred embodiments, the polynucleotide of such methods comprises a rabbit beta globin intron sequence comprising SEQ ID NO: 9 or a sequence having at least about 90% identity therewith, the poly(A) polynucleotide sequence is a BGH poly(A) polynucleotide sequence (SEQ ID NO: 4) or a polynucleotide sequence having at least about 90% identity therewith, the transgene comprises a coronavirus spike protein, and the number of Ad vector particles is at least about 30-350 times (e.g., preferably any of 30, 85 or 350 times) the number of Ad vector particles obtained from an Ad vector package cell lacking the TetR polynucleotide.

In some preferred embodiments, the polynucleotide used in such methods comprise a rabbit beta globin intron sequence comprising SEQ ID NO: 9 or a sequence having at least about 90% identity therewith, the poly(A) polynucleotide sequence is a BGH poly(A) polynucleotide sequence (SEQ ID NO: 4) or a sequence having at least about 90% identity therewith, the transgene comprises an influenza hemagglutinin (HA) surface protein antigen from A/Perth/16/2009(H3N2), and the number of Ad vector particles is at least any of about 5 to 15 times (preferably at least about 5 or 15) the number of Ad vector particles obtained from an Ad vector package cell lacking the TetR polynucleotide.

In some preferred embodiments, this disclosure provides adenoviral particle(s) prepared using polynucleotides and/or methods disclosed herein. In some preferred embodiments, this disclosure provides compositions, preferably an immunogenic composition comprising such adenoviral particle(s). In some preferred embodiments, this disclosure provides a vaccine comprising such composition. In some preferred embodiments, this disclosure provides methods for treating and/or preventing disease using such compositions and/or vaccines (or vaccine compositions), particularly in a mammalian subject (preferably a human being). One of skill in the art understands that an effective dose in a mouse may be scaled for larger animals such as a human, dogs, pigs, etc. In that way, through allometric scaling (also referred to as biological scaling) a dose in a larger animal may be extrapolated from a dose in a mouse to obtain an equivalent dose based on body weight or body surface area of the mammalian subject. In some embodiments, the methods comprise administering at least a prime and boost dose of a present immunogenic composition/formulation/dosage. In certain embodiments, the boost dose is administered about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks or 52 weeks, 2 years, 3 years, 4 years, 5 years, or more after administration of the prime dose; or after the boost dose (e.g., a second, third or more boost).

Pharmaceutically acceptable compositions may be solid compositions. The fluorocarbon-linked peptide composition may be obtained in a dry powder form. A cake resulting from lyophilization can be milled into powder form. A solid composition according to the invention thus may take the form of free-flowing particles. The solid composition typically is provided as a powder in a sealed vial, ampoule or syringe. If for inhalation, the powder can be provided in a dry powder inhaler. The solid matrix can alternatively be provided as a patch. A powder may be compressed into tablet form. The dried, for example, lyophilized, peptide or fluorocarbon-linked peptide composition may be reconstituted prior to administration. As used herein, the term “reconstitution” is understood to mean dissolution of the dried vaccine product prior to use. Following drying, such as lyophilization, the immunogenic peptide, for example, the fluorocarbon-linked peptide product, preferably is reconstituted to form an isotonic, PH neutral, homogeneous suspension. The formulation is typically reconstituted in the aqueous phase, for example by adding Water for Injection, histidine buffer solution (such as 28 mM L-histidine buffer), sodium bicarbonate, Tris-HCl or phosphate buffered saline (PBS). The reconstituted formulation is typically dispensed into sterile containers, such as vials, syringes or any other suitable format for storage or administration. The composition may be stored in a container, such as a sterile vial or syringe, prior to use.

The compositions/formulations can be administered in dosages and by techniques well known to those skilled in the clinical arts taking into consideration such factors as the age, sex, weight, and the route of administration. The formulations can be administered alone (i.e., as the sole active agent(s)) or can be co-administered or sequentially administered with compositions, e.g., with “other” immunogenic compositions or therapeutic compositions thereby providing multivalent or “cocktail” or combination compositions of the invention and methods employing them. In some embodiments, the formulations may comprise sucrose as a cryoprotectant and polysorbate-80 as a non-ionic surfactant. In certain embodiments, the formulations further comprise free-radical oxidation inhibitors ethanol and histidine, the metal-ion chelator ethylenediaminetetraacetic acid (EDTA), or other agents with comparable activity (e.g., block or prevent metal-ion catalyzed free-radical oxidation).

The compositions (e.g., formulations) may be present in a liquid preparation for mucosal administration, e.g., oral, nasal, ocular, etc., formulations such as suspensions and, preparations for parenteral, subcutaneous, intradermal, intramuscular, intravenous (e.g., injectable administration) such as sterile suspensions or emulsions. In such formulations the adenoviral vector may be in admixture with a suitable carrier, diluent, or excipient such as sterile water, physiological saline, viscosity enhancing excipients or the like. Certain specialized formulations for mucosal administration can be used, including mucoadhesives, mucosal penetrants and mucosal disruptants. The formulations can also be lyophilized or frozen. The formulations can contain auxiliary substances such as wetting or emulsifying agents, pH buffering agents, adjuvants, preservatives, and the like, depending upon the route of administration and the preparation desired. The formulations can contain at least one adjuvant compound. In exemplary embodiments, the present immunogenic compositions (e.g., vaccines) are non-adjuvanted. Standard texts, such as “REMINGTON'S PHARMACEUTICAL SCIENCE”, 17th edition, 1985, incorporated herein by reference, may be consulted to prepare suitable preparations, without undue experimentation.

In some preferred embodiments, this disclosure provides a recombinant polynucleotide comprising at least one Tetracycline operon repressor protein (TetR) expression cassette (TetR expression cassette), wherein each TetR expression cassette comprises: a CAG promoter/chimeric intron sequence that is optionally SEQ ID NO: 2; and, a poly(A) polynucleotide sequence that is optionally SEQ ID NO: 4 or SEQ ID NO: 6, operably linked to a coding sequence for the TetR coding sequence that is optionally SEQ ID NO: 3; optionally wherein the recombinant polynucleotide comprising a sequence having at least about 90% identity with SEQ ID NOs. 2, 3, 4 and/or 6. In some preferred embodiments, the TetR expression cassette comprises and/or is SEQ ID NO.: 1 or SEQ ID NO.: 5, or an expression cassette having at least about 90% identity therewith.

In some preferred embodiments, this disclosure provides a recombinant polynucleotide comprising at least one Tetracycline operon repressor protein (TetR) expression cassette that is optionally SEQ ID NO: 7 or an expression cassette having at least about 90% identity therewith; wherein each TetR expression cassette comprises a CMV promoter sequence that is optionally SEQ ID NO: 8 or a sequence having at least about 90% identity therewith, a rabbit beta-globin intron sequence that is optionally SEQ ID NO: 9 or a sequence having at least about 90% identity therewith, and a poly(A) polynucleotide sequence operably linked to a coding sequence for the TetR expression sequence is optionally SEQ ID NO: 3 or a sequence having at least about 90% identity therewith.

In some preferred embodiments, this disclosure provides a TetR expressing cell comprising TetR expression cassette integrated into the CAP cell genome. In preferred embodiments, the TetR expression cassette of such a cell is selected from the group consisting of SEQ ID NO: 1, SEQ ID NO: 5, SEQ ID NO: 7, and a TetR expression cassette having at least about 90% identity therewith. In some preferred embodiments, the cell is a human cell. In some preferred embodiments, the cell is an immortalized human amniocyte cell line.

In some preferred embodiments, this disclosure provides methods for producing an Ad vector(s) encoding a transgene encoding a toxic polypeptide, the method comprising obtaining a Tetracycline operon repressor protein (TetR) expressing cell comprising an exogenous polynucleotide in its genome, the polynucleotide comprising at least one Tetracycline operon repressor protein (TetR) expression cassette, each TetR expression cassette comprising a CAG promoter/chimeric intron sequence that is optionally SEQ ID NO: 2, and a poly(A) polynucleotide sequence that is optionally SEQ ID NO: 4 or SEQ ID NO: 6, operably linked to a coding sequence for the TetR gene that is optionally SEQ ID NO: 3; transfecting the TetR expressing cells with at least one Tetracycline operon operator (TetO) polynucleotide sequence linked to the promoter in a recombinant Ad genomic plasmid to produce a replication-deficient Ad vector containing at least one TetO polynucleotide sequence and encoding at least one transgene; and, isolating the Ad vector particles containing at least one TetO polynucleotide sequence and encoding at least one of such transgenes; wherein the TetR expression cassette of the cells comprises polynucleotides having at least about 90% identity with any of SEQ ID NOs. 2, 3, 4 or 6; and, the number of Ad vector particles containing at least one TetO polynucleotide sequence(s) and encoding at the least one transgene isolated from the cells comprising the TetR expression cassette(s) is at least about any of two, 10, 15, 20, 50, 100, 150, 200, 250, or 300 times the number of Ad vector particles obtained from an Ad vector package cell lacking the TetR polynucleotide. In some preferred embodiments, the TetR expression cassette of such cells comprises SEQ ID NO: 1 or SEQ ID NO: 5, or a TetR expression cassette(s) having at least about 90% identity therewith. In some preferred embodiments, the TetR expression cassette comprises a chimeric intron comprises a chicken beta actin intron sequence and a rabbit beta goblin intron sequence. In some preferred embodiments, the TetR expression cassette comprises a CAG/chimeric intron sequence according to SEQ ID NO: 2, or an intron sequence having at least about 90% identity therewith. In some preferred embodiments, the CAG/chimeric intron comprises SEQ ID NO: 2 or sequence having at least about 90% identity therewith, the poly(A) polynucleotide sequence is BGH (SEQ ID NO: 4) or a sequence having at least about 90% identity therewith, the transgene comprises a coronavirus spike protein, and the number of Ad vector particles is at least about any of 20, 60 or 200 times the number of Ad vector particles obtained from an Ad vector package cell lacking the TetR polynucleotide. In some preferred embodiments, the CAG/chimeric intron comprises SEQ ID NO: 2 or sequence having at least about 90% identity therewith, the poly(A) polynucleotide sequence is SV40 (SEQ ID NO: 6) or sequence having at least about 90% identity therewith, the transgene comprises a coronavirus spike protein, and the number of Ad vector particles is at least about any of 30, 150 or 200 times the number of Ad vector particles obtained from an Ad vector package cell lacking the TetR polynucleotide.

In some preferred embodiments, the TetR expression cassette comprises at least one CAG/chimeric intron comprises SEQ ID NO: 2 or sequence having at least about 90% identity therewith, the poly(A) polynucleotide sequence is a BGH poly(A) polynucleotide sequence (SEQ ID NO: 4) or sequence having at least about 90% identity therewith, the transgene comprises an influenza hemagglutinin (HA) surface protein antigen from A/Perth/16/2009(H3N2), and the number of Ad vector particles is at least any of about two, 10, or 20 times the number of Ad vector particles obtained from an Ad vector package cell lacking the TetR polynucleotide. In some preferred embodiments, the CAG/chimeric intron comprises SEQ ID NO: 2 or sequence having at least about 90% identity therewith, the poly(A) polynucleotide sequence is a SV40 poly(A) polynucleotide sequence (SEQ ID NO: 6) or sequence having at least about 90% identity therewith, the transgene comprises an influenza hemagglutinin (HA) surface protein antigen from A/Perth/16/2009(H3N2), and the number of Ad vector particles is at least any of about five or 10 times the number of Ad vector particles obtained from an Ad vector package cell lacking the TetR polynucleotide.

In some preferred embodiments, this disclosure provides methods for producing an adenoviral vector encoding a transgene, the method comprising: obtaining a Tetracycline operon repressor protein (TetR) recombinant cell comprising an exogenous polynucleotide in its genome, the polynucleotide comprising at least one Tetracycline operon repressor protein (TetR) expression cassette, each TetR expression cassette comprising a CMV promoter sequence (SEQ ID NO: 8), a rabbit beta globin intron sequence (SEQ ID NO: 9), and a poly(A) polynucleotide sequence (SEQ ID NO: 4) operably linked to a coding sequence for the TetR gene (SEQ ID NO: 3); transfecting the TetR expressing cells with at least one Tetracycline operon operator (TetO) polynucleotide sequence linked to the promoter in a recombinant Ad genomic plasmid to a replication-deficient Ad vector containing at least one TetO polynucleotide sequence and encoding at least one transgene; and, isolating the Ad vector particles containing at least one TetO polynucleotide sequence and encoding at least one transgene; wherein the TetR expressing cells comprise at least one polynucleotide having at least about 90% identity with any of SEQ ID NOs. 3, 4 8, or 9; and, the number of Ad vector particles containing at least one TetO polynucleotide sequence and encoding at least one such transgene isolated from cells comprising the at least one TetR expression cassettes is at least about any of two, 10, 15, 20, 50, 100, 150, 200, 250, or 300 times the number of Ad vector particles obtained from an Ad vector package cell lacking the TetR expression cassette. In some preferred embodiments, the TetR expression cassette of such cells is according to SEQ ID NO: 7 or an expression cassette have at least about 90% identity therewith. In some preferred embodiments, the intron sequence of the TetR expression cassette(s) is SEQ ID NO: 9 or an intron sequence having at least about 90% identity therewith.

In some preferred embodiments, the rabbit beta globin intron sequence of the TetR expression cassette comprises SEQ ID NO: 09 or a sequence having at least about 90% identity therewith, a poly(A) polynucleotide sequence that is a BGH poly(A) polynucleotide sequence (preferably SEQ ID NO: 4) or a polynucleotide sequence having at least about 90% identity therewith, the transgene comprises a coronavirus spike protein, and the number of Ad vector particles is at least about any of 30, 85 or 350 times the number of Ad vector particles obtained from an Ad vector package cell lacking the TetR polynucleotide.

In some preferred embodiments, the rabbit beta globin intron sequence of the TetR expression cassette comprises a rabbit beta globin intron sequence comprises SEQ ID NO: 9 or a sequence having at least about 90% identity therewith, the poly(A) polynucleotide sequence is a BGH poly(A) polynucleotide sequence (preferably SEQ ID NO: 4) or a sequence having at least about 90% identity therewith, the transgene comprises an influenza hemagglutinin (HA) surface protein antigen from A/Perth/16/2009(H3N2), and the number of Ad vector particles is at least any of about 5 or 15 times the number of Ad vector particles obtained from an Ad vector packaging cell lacking the TetR polynucleotide.

In some preferred embodiments, the TetR expression cassettes of this disclosure and used in the methods of this disclosure comprise at least one poly(A) polynucleotide sequence is a BGH or SV40 poly(A) polynucleotide sequence, optionally SEQ ID NO: 4 or SEQ ID NO: 6, or a polynucleotide sequence having at least about 90% identity therewith.

In some preferred embodiments, the transgene encodes a viral or bacterial antigen. In some preferred embodiments, the viral antigen is a coronavirus or flu antigen. In some preferred embodiments, this disclosure provides an adenoviral particle prepared using any of the reagents and/or methods disclosed herein.

Other embodiments are also contemplated herein as would be understood by those of ordinary skill in the art.

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

The compositions, formulations and methods of the present invention may comprise, consist essentially of, or consist of the components and ingredients of the present invention as well as other ingredients described herein. As used herein, “consisting essentially of” means that the compositions, formulations and methods may include additional steps, components or ingredients, but only if the additional steps, components or ingredients do not materially alter the basic and novel characteristics of the claimed compositions, formulations and methods.

In certain embodiments, non-invasive administration of the immunogenic (preferably anti-virus) composition, includes, but is not limited to, topical application to the skin, and/or intranasal and/or mucosal and/or perlingual and/or buccal and/or oral and/or oral cavity and/or intramuscular administration. Dosage forms for the application of the immunogenic (preferably anti-virus) composition may include liquids, ointments, powders and sprays. The active component may be admixed under sterile conditions with a physiologically acceptable carrier and any preservative, buffers, propellants, or absorption enhancers as may be needed.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

As used herein, an “adjuvant” refers to a substance that enhances the body's immune response to an antigen. In embodiments, the present monovalent influenza pharmaceutical formulation is a non-adjuvanted vaccine composition.

By “administration” is meant introducing a vaccine composition of the present disclosure into a subject; it may also refer to the act of providing a composition of the present disclosure to a subject (e.g., by prescribing).

As used herein, the term “ambient temperature” is the air temperature for storing the present monovalent influenza pharmaceutical formulation. In embodiments, the ambient temperature is a room temperature, such as selected from any temperature within the range from about 15 to 30° C., preferably from about 20 to 25° C.

The term “therapeutically effective amount” as used herein refers to that amount of the compound being administered which will induce a combined, mucosal, humoral and cell mediated immune response. The term also refers to an amount of the present compositions that will relieve or prevent to some extent one or more of the symptoms of the condition to be treated. In reference to conditions/diseases that can be directly treated with a composition of the disclosure, a therapeutically effective amount refers to that amount which has the effect of preventing the condition/disease from occurring in a mammal that may be predisposed to the disease but does not yet experience or exhibit symptoms of the condition/disease (prophylactic treatment), alleviation of symptoms of the condition/disease, diminishment of extent of the condition/disease, stabilization (e.g., not worsening) of the condition/disease, preventing the spread of condition/disease, delaying or slowing of the condition/disease progression, amelioration or palliation of the condition/disease state, and combinations thereof. The term “effective amount” refers to that amount of the compound being administered which will produce a reaction that is distinct from a reaction that would occur in the absence of the compound.

As used herein, the term “percent (%) homology” and grammatical variations thereof in the context of two sequences (e.g., protein sequences), refers to two or more sequences or subsequences (i.e., fragment thereof) that have at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity (homology), when compared and aligned for maximum correspondence, as measured using one of the well-known sequence comparison algorithms or by visual inspection.

As used herein, an “immunogenic composition” refers to a composition, typically comprising at least one type of peptide construct as disclosed herein and at least one pharmaceutically acceptable carrier that, when administered to a host induces, stimulates, and/or enhances an immune response against at least one type of virus (e.g., SARS-CoV-2 or influenza) antigen(s). A “vaccine” refers to such an immunogenic composition that when administered induces, stimulates, and/or enhances a protective immune response against such a virus (e.g., SARS-CoV-2 or influenza) virus (e.g., protects the host against challenge with coronavirus (e.g., SARS-CoV-2 or influenza). In certain embodiments, an immunogenic composition (e.g., vaccine) can comprise one or more peptide constructs comprising at least one coronavirus antigen and/or influenza antigen(s), along with other components of an immunogenic composition (e.g., vaccine) suitable for administration to a mammalian host, including for example one or more adjuvants, slow release compounds, solvents, buffers, additional anti-coronavirus and/or anti-influenza agents, etc. In certain embodiments, an immunogenic composition and/or vaccine can comprise a protein and/or carbohydrate and/or lipid and/or other antigen, including but not limited to one or more killed antigen(s) (e.g., a killed or completely inactive virus) or a live attenuated antigen (e.g., an attenuated virus). In some embodiments, the immunogenic composition(s) and/or vaccine(s) improve immune responses to any antigen regardless of the antigen source or its function.

As used herein, a “pharmaceutically acceptable carrier” refers to a carrier or diluent that does not cause significant irritation to the human subject and does not abrogate the biological activity and properties of the administered vaccine compositions.

As used here, the term “seroconversion” is defined as a four-fold or greater increase in serum neutralization antibody titers (e.g., a sufficient quantity of antibodies in serum that can neutralize an infectious agent) after vaccination (e.g., administration of a present immunogenic composition).

As used herein, the term “seropositive” means a measurable (e.g., detectable in an in vitro assay) in serum neutralization antibody after vaccination (e.g., administration of a present immunogenic composition).

As used herein, the term “seroprotected” means a subject post vaccination that is protected from infection via generation of serum neutralization antibodies. In a population, this is referred to as a percentage (%) of seroprotected individuals (e.g., 50%). In embodiments, the present immunogenic compositions and methods of use provide seroprotection to the mammalian subject, such as a human subject, against a viral infection (e.g., SARS-CoV-2 or influenza).

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also mean prolonging survival as compared to expected survival if not receiving treatment and/or can be therapeutic in terms of a partial or complete cure for a disease and/or adverse effect attributable to the disease. As used herein, the terms “prophylactically treat” or “prophylactically treating” refers completely, substantially, or partially preventing a disease/condition or one or more symptoms thereof in a host. Similarly, “delaying the onset of a condition” can also be included in “prophylactically treating” and refers to the act of increasing the time before the actual onset of a condition in a patient that is predisposed to the condition.

As used herein, a “vaccine” refers to a composition comprising an Ad vector that can be used to induce an anti-virus immune response, along with other components of a vaccine formulation, including for example adjuvants, slow-release compounds, solvents, etc. In embodiments of the invention, vaccines improve immune responses to any antigen regardless of the antigen source or its function.

As referred to herein an “antigen” means a substance that induces and/or enhances a specific immune response against the antigen, and/or an infectious agent expressing such antigen, in a subject, including humans and/or animals. The antigen may comprise an epitope, a hapten, and/or any combination thereof.

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g., amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Generation of Stably Transfected CAP-TetR Cell Line (Z3634) Based on TetR Expression Cassette #1

Stably transfected CAP-TetR cell line (Z3634) were obtained following the transfection of the parental CAP cells with a plasmid containing “TetR expression cassette #1” (SEQ ID NO: 1) described in FIG. 1. The TetR expression cassette #1 (SEQ ID NO.: 1) comprises a CAG promoter/Chimeric intron sequence, (SEQ ID NO.: 2) a Tetracycline Repressor protein (TetR) sequence and (SEQ ID NO.: 3) a poly A sequence from the Bovine Growth Hormone (BGH) (SEQ ID NO.: 4). Briefly, the TetR expression cassette #1 was incorporated into a pStbl vector to produce plasmids p1085 which was linearized and transfected into CAP cells before antibiotic selection and expansion for the selection of a stably transfected cell pools (see Table 1 below (e.g., CAP-TetR cell line Z3634 produced using the p1085 plasmid).

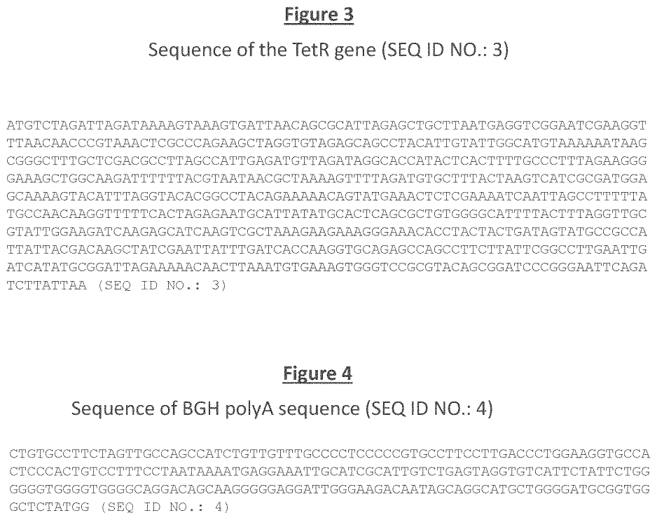

The CAG promoter/Chimeric intron sequence (SEQ ID NO: 2) comprises four components: (1) the CMV early enhancer/promoter element; (2) the first exon and the first intron of chicken beta-actin gene; (3) the splice acceptor of the rabbit beta-globin gene and (4) the chimeric intron comprises a combination of an intron sequence from chicken beta actin and rabbit beta globin as described separately in FIG. 2. The sequence of TetR gene (SEQ ID NO: 3) is described separately in FIG. 3. The sequence of BGH polyA sequence (SEQ ID NO: 4) is described separately in FIG. 4.

For the generation of the Z3634 CAP-TetR cell pool generation, the P1085 TetR expression cassette #1 (FIG. 1) plasmid was linearized followed by phenol-chloroform extraction and ethanol precipitation. Parental CAP cells were thawed and cultivated in Protein Expression Medium (PEM) supplemented with 4 mM GlutaMax (PEM+). Nucleofection and pool generation were performed as described in the following. During exponential growth phase of the culture, the CAP cells were counted and viable cell density and viability were determined. For each nucleofection reaction 1×10⁷ viable cells were harvested by centrifugation (300-500 g, 5 min). The cells were resuspended in 100 μl complete nucleofector solution V and mixed with 5 μg of the linearized P1085 TetR expression cassette #1 plasmid DNA. The DNA/cell suspension was transferred into a cuvette and the nucleofection was performed using the internal Standard Operating Procedure (SOP). After the pulse, cells were recovered by adding 500 μl pre-warmed PEM+ to the cuvette and gently transferred into 11.5 ml PEM+ in a 125 ml shake flask or in a 50 ml SpinTube. The cuvette was washed once with 500 μl fresh PEM+ to recover residual cells.

The cells were cultured as at 37° C., 5% CO₂ at 120 rpm, 5 cm orbit (shake flask) or at 37° C., 5% CO₂ at 185 rpm, 5 cm orbit (SpinTube). One passage post transfection, 5 μg/ml Blasticidin was added for selection of the pool. Six to eight cryovials of cells from the stable pools were frozen in 50% complete growth medium, 7.5% DMSO and 42.5% ProFreeze (1.5×10⁷ cells, 1.8 ml per vial).

### Example 2: Generation of Stably-Transfected CAP-TetR Cell Line (Z3635) Based on TetR Expression Cassette #2 (p1086)

Stably-transfected CAP-TetR cell line (Z3635) was obtained following the transfection of the parental CAP cells with a plasmid containing “TetR expression cassette #2” (SEQ ID NO: 5) described in FIG. 5. The TetR expression cassette #2 comprises (1) a CAG promoter/Chimeric intron sequence, (2) a Tetracycline Repressor protein (TetR) sequence and (3) a SV40 polyA sequence. Briefly, the TetR expression cassette #2 was incorporated into a pStbl vector to generate plasmid p1086 which was linearized and transfected into CAP cells before antibiotic selection and expansion for the selection of a stably transfected cell pools (CAP-TetR cell line Z3635).

The CAG promoter/Chimeric intron sequence (SEQ ID NO: 2) and the TetR gene sequence (SEQ ID NO: 3) have been described in Example 1. The SV40 polyA sequence (SEQ ID NO: 6) is described separately in FIG. 6. The Z3635 CAP-TetR cell pool generation was performed by nucleofection of the p1086 TetR expression cassette #2 plasmid according to the methodology described in Example 1.

### Example 3: Generation of Stably-Transfected CAP-TetR Cell Line (C235) Based on TetR Expression Cassette #3 (p1116)

Stably-transfected CAP-TetR cell line (C235) was obtained following the transfection of the parental CAP cells with a plasmid containing “TetR expression cassette #3” (SEQ ID NO: 7) described in FIG. 7. The TetR expression cassette #3 comprises (1) a CMV promoter sequence, (2) a β-Globin intron sequence (3) a Tetracycline Repressor protein (TetR) sequence and (4) a BGH polyA sequence. Briefly, the TetR expression cassette #3 was incorporated into a pStbl vector to generate plasmid p1116 which was linearized and transfected into CAP cells before antibiotic selection and expansion for the selection of a stably transfected cell pools (CAP-TetR cell line C235).

The CMV promoter sequence (SEQ ID NO: 8) is described separately in FIG. 8. The β-Globin intron sequence (SEQ ID NO: 9) is described separately in FIG. 9. The TetR gene sequence (SEQ ID NO: 3) has been described in Example 1. The bovine growth hormone (BGH) poly A (SEQ ID NO: 4) has been described in Example 1.

The C235 CAP-TetR cell pool generation was performed by nucleofection of the p1116 TetR expression cassette #3 plasmid according to the methodology described in Example 1.

### Example 4: Generation of Stably-Transfected CAP-TetR Cell Lines Z3616 Based Respectively on TetR Expression Cassette #4

Based on a similar method as described in Examples 1 to 3, the stably-transfected CAP-TetR cell line (Z3616) was obtained following the transfection of the parental CAP cells with a plasmid containing “TetR expression cassette #4” (SEQ ID NO: 10) described in FIG. 10. The TetR expression cassette #4 comprises (1) a CAG promoter sequence, (2) a SV40 intron sequence (3) a Tetracycline Repressor protein (TetR) sequence and (4) a BGH polyA sequence. Briefly, the TetR expression cassette #4 was incorporated into a pStbl vector to generate p1058 plasmid which was linearized and transfected into CAP cells before antibiotic selection and expansion for the selection of a stably transfected cell pools (CAP-TetR cell line Z3616). The Z3616 CAP-TetR cell pool generation was performed by nucleofection of the TetR expression cassette #4 plasmid p1058 according to the methodology described in Example 1.

### Example 5: Generation of Stably-Transfected CAP-TetR Cell Lines Z3617 Based Respectively on TetR Expression Cassette #5

Based on a similar method as described in Examples 1 to 3, a stably-transfected CAP-TetR cell line (Z3617) was obtained following the transfection of the parental CAP cells with a plasmid containing “TetR expression cassette #5” (SEQ ID NO: 11) described in FIG. 11. The TetR expression cassette #5 comprises (1) a CAG promoter sequence, (2) a SV40 intron sequence (3) a Tetracycline Repressor protein (TetR) sequence and (4) a SV40 polyA sequence. Briefly, the TetR expression cassette #5 was incorporated to a pStbl vector to generate p1059 plasmid which was linearized and transfected into CAP cells before antibiotic selection and expansion for the selection of a stably transfected cell pools (CAP-TetR cell line Z3617). The Z3617 CAP-TetR cell pool generation was performed by nucleofection of the TetR expression cassette #5 plasmid p1059 according to the methodology described in Example 1.

### Example 6: Generation of Stably-Transfected CAP-TetR Cell Lines Z3618 Based Respectively on TetR Expression Cassette #6

Based on a similar method as described in Examples 1 to 3, stably-transfected CAP-TetR cell line (Z3618) were obtained following the transfection of the parental CAP cells with a plasmid containing “TetR expression cassette #6” (SEQ ID NO: 12) described in FIG. 12. The TetR expression cassette #6 comprises (1) a CMV promoter sequence, (2) a SV40 intron sequence (3) a Tetracycline Repressor protein (TetR) sequence and (4) a BGH polyA sequence. Briefly, the TetR expression cassette #6 was incorporated into a pStbl vector to generate p1060 plasmid which was linearized and transfected into CAP cells before antibiotic selection and expansion for the selection of a stably transfected cell pools (CAP-TetR cell line Z3618). The Z3618 CAP-TetR cell pool generation was performed by nucleofection of the TetR expression cassette #6 plasmid p1060 according to the methodology described in Example 1.

### Example 7: Generation of Stably-Transfected CAP-TetR Cell Lines C236 Based Respectively on TetR Expression Cassette #7

Based on a similar method as described in Examples 1 to 3, the stably-transfected CAP-TetR cell line (C236) was obtained following the transfection of the parental CAP cells with a plasmid containing “TetR expression cassette #7” (SEQ ID NO: 13) described in FIG. 13. The TetR expression cassette #7 comprises (1) a CMV promoter sequence, (2) a Tetracycline Repressor protein (TetR) sequence and (3) a BGH polyA sequence. Briefly, the TetR expression cassette #7 was incorporated into a pStbl vector to generate p1117 plasmid which was linearized and transfected into CAP cells before antibiotic selection and expansion for the selection of a stably transfected cell pools (CAP-TetR cell line C236). The C236 CAP-TetR cell pool generation was performed by nucleofection of the TetR expression cassette #7 plasmid p1117 according to the methodology described in Example 1.

### Example 8: Expression of TetR in the Generated CAP-TetR Cell Line

Based on the different expression cassettes described in example 2 to 8, the seven respective cell lines were tested for the successful expression of TetR protein. Surprisingly, only cell lines Z3634, Z3635 and C235 were able to express detectable levels of TetR as presented in FIGS. 14-16 and summarized in Table 1.

TABLE 1
Tet Expression across the different CAP-TetR Cell Line generated

| Cassette | | | | | Cell Line | TetR |
|---|---|---|---|---|---|---|
| number | Identifier | Promoter | Intron | Poly(A) | Pool | Expression |
| #1 | p1085 | CAG | Chimeric | BGH | Z3634 | Yes |
| #2 | p1086 | CAG | Chimeric | SV40 | Z3635 | Yes |
| #3 | p1116 | CMV | Beta-globin | BGH | C235 | Yes |
| #4 | p1058 | CAG | SV40 | BGH | Z3616 | No |
| #5 | p1059 | CAG | SV40 | SV40 | Z3617 | No |
| #6 | p1060 | CMV | SV40 | BGH | Z3618 | No |
| #7 | p1117 | CMV | None | BGH | C236 | No |

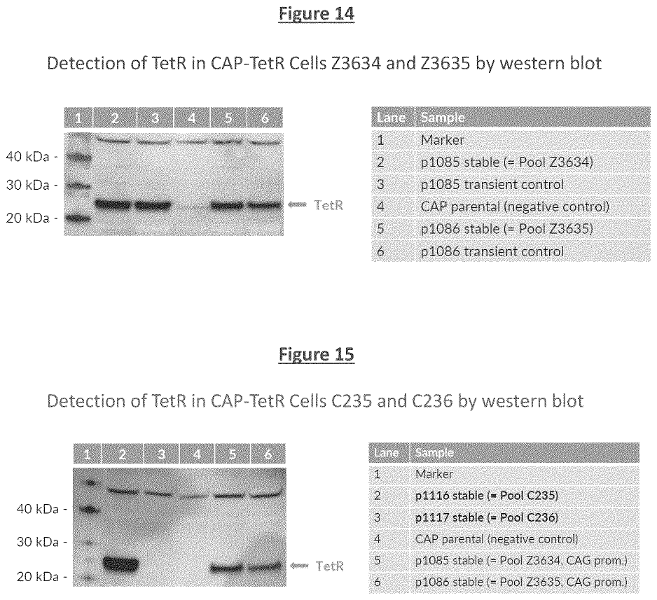

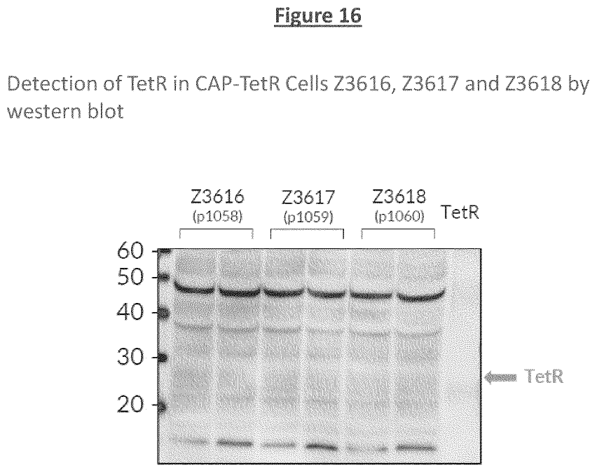

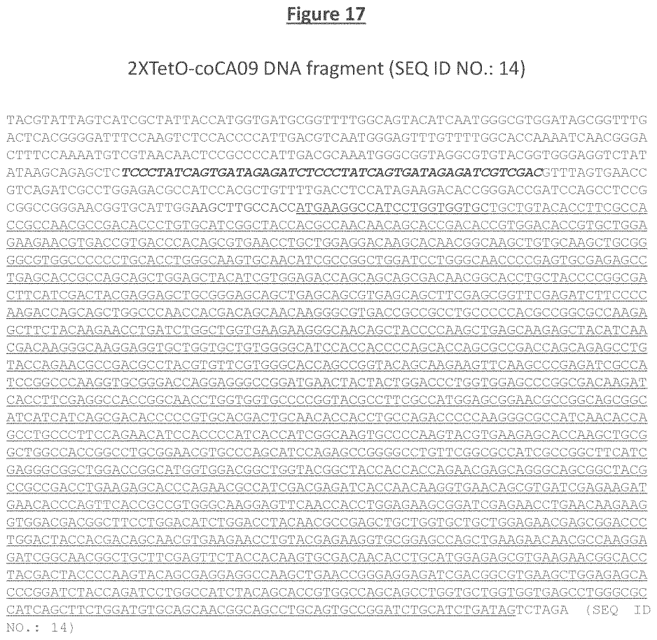

Detection of TetR protein expression was performed by Western Blot. Briefly, cultured cells were centrifuged and lysed with RIPA buffer. A sample of 25 μl (equivalent to 1.8E04 lysed cells/lane) was loaded a 4-12% NuPage gel. After completion of the electrophoresis, the gel was transferred to a PVDF membrane. Detection of TetR was performed in two steps (1) use of an anti-TetR mAb (MoBiTec, cat #TET02, dilution 1:500 (v/v) followed by (2) the use of a secondary antibody Anti-mouse IgG, HRP-linked (CST, cat #7076, dilution 1:50000 (v/v)). TetR positive bands were detected using the Super Signal West Atto substrate. Results for CAP-TetR Cells Z3634 and Z3635 are shown in FIG. 14. Results for CAP-TetR Cells C235 and C236 are presented in FIG. 15. Results for cells CAP-TetR Cells Z3616, Z3617 and Z3618 are presented in FIG. 16.

### Example 9: Construction and Generation of Ad2×TetO-Vectors (Containing Tetracycline Operator) and Corresponding Ad Vector Control (without Tetracycline Operator)

The generation of Ad2×TetO-Vectors (Ad vectors with TetO) is briefly summarized as follows. The first step in the process consist in the construction of a pAdhigh shuttle plasmid which contains a desired transgene DNA fragment and a cytomegalovirus (CMV) promoter comprises two copies of Tetracycline operator (TetO) sequence (pAdhigh2×TetO-shuttle). The pAdhigh2×TetO-shuttle is then linearized by Pmel digestion before being transformed BJ5183-AD-1 electroporation competent cells which contain pAdEasy-1 plasmid (Agilent) to create a recombinant Ad genomic plasmid (pAd2×TetO-vector) containing the desired transgene. Following amplification and purification, the resulting pAd2×TetO-vector plasmid is linearized by PacI digestion before transfection into Ad vector packaging cells such as CAP or CAP-TetR cells to produce an Ad2×TetO-Vector.

Example of the shuttle plasmid containing CMV early promoter, two copies of TetO sequence and the sequence for the hemagglutinin (HA) antigen derived from influenza A(H1N1) pdm09 virus is described below. Briefly, a DNA fragment (2×TetO-coCA09; SEQ ID NO: 14) was synthesis by Genscript containing two copies of the TetO sequence (TCCCTATCAGTGATAGAGA) and the codon-optimized sequence of HA coCA09 (FIG. 17).

The synthetic 2×TetO-coCA09 HA DNA fragment (SEQ ID NO: 14) was subsequently cloned into SnaBI and XbaI sites in Altimmune's pAdhighSwaI vector by GenScript to generated pAdhigh2×TetO-coCA09 shuttle vector for Altimmune.

All other pAdhigh2×TetO-shuttle vectors are generated similarly by replacing coCA09 DNA fragment in the pAdhigh2×TetO-coCA09 shuttle vector with a desired transgene DNA fragment. Specifically, pAdhigh2TetO-tPAWHS was generated encoding codon-optimized full length spike protein from the SARS-CoV-2 Wuhan strain with a tissue plasminogen activator (tPA) signal sequence (tPAWHS); and pAdhigh2×TetO-coPerth was generated encoding a codon-optimized Perth HA gene from A/Perth/16/2009(H3N2) (coPerth).

pAdhigh2TetO-tPAWHS and pAdhigh2×TetO-coPerth shuttle plasmids were then linearized by Pmel digestion before being transformed BJ5183-AD-1 electroporation competent cells to respectively generate the recombinant Ad genomic plasmids pAd2TetO-tPAWHS and pAd2×TetO-coPerth.

Control recombinant Ad genomic plasmids pAdtPAWHS and pAdcoPerth, respectively, encoding tPAWHS and coPerth (as described above) were generated without TetO sequence(s) in their CMV promoters. An additional Ad vector AdE was also generated without TetO sequence(s) without any transgene. Briefly, codon-optimized tPAWHS DNA fragment was synthesized and cloned into pAdhighSwaI shuttle vector to generate pAdhightPAWHS shuttle plasmids. Codon-optimized Perth HA was synthesized and cloned into pAdhigh shuttle vector to generate pAdhighcoPerth shuttle plasmid. The pAdhightPAWHS and pAdhighcoPerth shuttle plasmids were digested with Pmel and transformed into BJ5183-AD-1 electroporation competent cells to generate pAdtPAWHS and pAdcoPerth recombinant Ad genomic plasmids.

Viral seeds of the different Ad2×TetO-Vectors (containing Tetracycline operator) and corresponding Ad vector control (without Tetracycline operator) were generated by electroporation (EP) according to an Altimmune Inc EP protocol. Briefly, the day before EP, CAP-TetR cell pools Z3634 (Example 2), Z3635 (Example 3), or C235 (Example 4) were centrifuged at 1100±100 rpm for 10 minutes at room temperature and the cell pellets were resuspended with serum-free AEM media supplemented with L-glutamine 4 mM (complete medium) to reach the cell concentration at 1×10⁶ cells/ml. The CAP-TetR (Z3634, Z3635 and C235) cells were cultured in a cell culture incubator at 37° C., 5% CO₂ with 120 rpm agitation for around 24 hours. For EP, the CAP-TetR (Z3634, Z3635 and C235) cells were centrifuged at 1100±100 rpm for 10 minutes at room temperature and resuspended in EP buffer at the concentration of 1×10⁸ alive cells/ml. 0.4 ml of cell suspension was mixed with 80 μg of PacI cut recombinant Ad genomic plasmids.

### Example 10: Improved Generation of Seed Ad2×TetO-Vectors in CAP-TetR Cells in (Z3634, Z3635, and C235) Compared to Corresponding Ad Vector Controls (without Tetracycline Operator)

Two transfection studies (EP38 and EP39) were performed with pAdtPAWHS, pAd2×TetO-tPAWHS, pAdcoPerth, and pAd2×TetO-coPerth respectively using CAP-TetR Cells Z3634 (EP38), C235 (EP39) and Z3635 (EP39).

The Z3634, Z3635, and C235 cells and linearized plasmid DNA (pAdtPAWHS, pAd2×TetO-tPAWHS, pAdcoPerth, and pAd2×TetO-coPerth) mixture underwent a static EP according to the manufacturing recommendation using a single-use processing assembly OC-400 and Maxcyte STX-100 instrument for rapid, high titer viral vector production. After EP, transfected CAP-TetR cells were incubated 30 minutes at 37° C. with DNase I to digest unincorporated plasmid DNA. Thirty (30) ml complete medium was added to transfected cells and incubated in a cell culture incubator at 37° C., 5% CO₂ with agitation for six days before to be harvested and processed to generate viral seed stock. Approximately 80% of cell culture media was changed three days post-EP. The cell count and viability were monitored during the six days.

The adenoviral vector harvesting was followed using the following protocol. Briefly, transfected or infected cells were collected by centrifugation at 1400±100 rpm for 15±1 minutes at ambient temperature. After supernatant aspiration, cell pellet(s) were resuspended with one tenth original volume of complete medium. Three freeze-thaw cycles of the harvested cells using a −80° C. freezer and 37° C. water bath was carried out. After three cycles of freeze/thaw, the cell lysate was centrifuged at 5000±100 rpm at 4° C. for 15±1 minutes. Supernatant(s) were collected and filtered using GP 0.22 μm filters. The filtered supernatant was the adenovirus vector stock which would be used for further adenoviral vector amplification and adenoviral vector viral titer assay.

The adenoviral vector titers were determined according to an Altimmune Inc approved protocol. Briefly, HEK-293 adherent cells were seeded, the day before the infection, in 96 wells plates at 45000 to 50000 cells/well. On the infection day, series of 10-fold dilutions of the virus sample were made in fresh RPMI 1640 media supplemented with L-glutamine 2 mM and 2% of fetal bovine serum. An adenovirus null vector (AdE) was used as internal positive standard control. Cell culture media on 96-wells plates was removed and replaced by 100 μl of virus sample dilution. Duplicates were performed for each sample. Plates were then incubated at 37° C., 5% CO₂ in a humidified environment. After three days post-infection, the infectious foci were visualized using the Adeno-X Rapid Titer Kit. The infectious forming unit (ifu) was determined according to an Altimmune Inc approved protocol.

The results of viral titers resulting from electroporation EP38 and EP39 are respectively presented in Table 2 and Table 3.

TABLE 2
EP38 on Z3634 (chimeric intron /BGH PolyA) CAP-TetR cells

| Viral vector | Viral titer (ifu/ml) | Structure |
|---|---|---|
| AdtPAWHS | Not detected | Spike SARS-Cov-2 |
| Ad2xTetO-tPAWHS | 1.04E+09 | Spike SARS-Cov-2 + TetO |
| AdcoPerth | 9.40E+07 | HA Influenza H3N2 |
| Ad2xTetO-coPerth | 2.67E+09 | HA influenza H3N2 + TetO |

TABLE 3
EP39 on Z3635 (chimeric intron/SV40 PolyA) and C235
(beta-globin intron/BGH PolyA) CAP-TetR cells

| | Viral titer (ifu/ml) | |
|---|---|---|
| Viral vector | Z3635 CAP-TetR | C235 CAP-TetR |
| AdtPAWHS | Not detected | Not detected |
| Ad2xTetO-tPAWHS | 1.23E+08 | 4.13E+06 |
| AdcoPerth | Not detected | Not detected |
| Ad2xTetO-coPerth | 1.15E+09 | 6.15E+06 |

These results demonstrated that Ad vectors encoding toxic transgenes could be generated with high viral titers in CAP-TetR cells when the promoters of the Ad vectors contain the TetO sequence as opposed to comparator Ad vectors without TetO sequence that could not be rescued or with low viral titer.

### Example 11: Higher Viral Yields Achieved with Ad2×TetO-Vectors Following Infection of Different CAP-TetR Cells Compared to Parental CAP Cells

For comparation of the viral production capability of adenovirus vaccine vectors expression of toxic transgenes, parental CAP (pCAP) cells and CAP-TetR cells were infected with Ad vector seeds generated above. An AdE vector was used as an infection control.

Infection of parental CAP (pCAP) cells and CAP-TetR cells was performed according to an Altimmune Inc. approved protocol. Briefly, the day before infection, cells were centrifuged at 1100±100 rpm for 10 minutes at room temperature and the cell pellets were resuspended with complete medium to reach the cell concentration at 1×10⁶ cells/ml. Cells were cultured in a cell culture incubator at 37° C., 5% CO₂ with 120 rpm agitation for around 24 hours. The appropriate volume of adenoviral vector seeds was thawed and added to the CAP or the CAP-TetR cell cultures. Infected cells were then incubated at 37° C., 5% CO₂ in a cell culture incubator with 120 rpm agitation for 3-5 days before to be harvested and processed to generate a new adenoviral vector stock. The cell count and viability were monitored during culture time.

In the first infection experiment, parental CAP, C235, Z3634, and Z3635 CAP-TetR cells were infected with Ad vectors Ad2×TetO-tPAWHS and Ad2×TetO-coPerth (generated from EP38 on Z3634 CAP-TetR cells) at an MOI of three (3). After infection, the cell culture was maintained for three (3) days. The results are summarized in Tables 4 (viral titers) and Table 5 (viral yield).

TABLE 4
Viral titers on parental CAP cells and CAP-TetR cells

| | Viral titer (ifu/ml) | | | |
|---|---|---|---|---|
| Viral vector | pCAP | Z3634 | Z3635 | C235 |
| Ad2xTetO-tPAWHS | 3.26E+04 | 7.13E+06 | 5.11E+06 | 2.88E+06 |
| Ad2xTetO-coPerth | 2.42E+06 | 5.69E+07 | 2.86E+07 | 4.07E+07 |
| AdE | 1.70E+08 | 3.11E+08 | 2.79E+08 | 2.68E+08 |

TABLE 5
Viral yield comparison between parental CAP
cells and CAP-TetR cells

| | Z3634 | | Z3635 | | C235 | |
|---|---|---|---|---|---|---|
| Viral vector | versus pCAP | | versus pCAP | | versus pCAP | |
| Ad2xTetO-tPAWHS | 218.7 | X | 156.7 | X | 88.3 | X |
| Ad2xTetO-coPerth | 23.5 | X | 11.8 | X | 16.8 | X |
| AdE | 1.83 | X | 1.64 | X | 1.58 | X |

In a second infection experiment, parental CAP, C235, Z3634, and Z3635 CAP-TetR cells were infected with Ad vectors Ad2×TetO-tPAWHS and Ad2×TetO-coPerth (generated from EP39 on Z3635 CAP-TetR cells) at an MOI of two (2). After infection, the cell culture was maintained for four (4) days. The results are summarized in Tables 6 (viral titers) and Table 7 (viral yields).

TABLE 6
Viral titers on parental CAP cells and CAP-TetR cells

| | Viral titer (ifu/ml) | | | |
|---|---|---|---|---|
| Viral vector | pCAP | Z3634 | Z3635 | C235 |
| Ad2xTetO-tPAWHS | 2.44E+05 | 5.38E+06 | 7.12E+06 | 6.88E+06 |
| Ad2xTetO-coPerth | 6.81E+06 | 7.10E+07 | 7.04E+07 | 5.34E+07 |
| AdE | 8.80E+08 | 4.60E+08 | 6.55E+08 | 5.40E+08 |

TABLE 7
Viral yield comparison between parent CAP cells
and CAP-TetR cells

| | Z3634 | | Z3635 | | C235 | |
|---|---|---|---|---|---|---|
| Viral vector | versus pCAP | | versus pCAP | | versus pCAP | |
| Ad2xTetO-tPAWHS | 22.0 | X | 29.2 | X | 28.2 | X |
| Ad2xTetO-coPerth | 10.4 | X | 10.3 | X | 7.84 | X |
| AdE | 0.52 | X | 0.74 | X | 0.61 | X |

In the third infection experiment, parental CAP (pCAP), C235, Z3634, and Z3635 CAP-TetR cells were infected with Ad vectors Ad2×TetO-tPAWHS and Ad2×TetO-coPerth (generated from EP39 on C235 CAP-TetR cells) at an MOI of 0.05. After infection, the cell culture was maintained for five (5) days. The results are summarized in Tables 8 and 9.

TABLE 8
Viral titers on parental CAP cells and CAP-TetR cells

| | Viral titer (ifu/ml) | | | |
|---|---|---|---|---|
| Viral vector | pCAP | Z3634 | Z3635 | C235 |
| Ad2xTetO-tPAWHS | 4.26E+04 | 2.76E+06 | 8.55E+06 | 1.47E+07 |
| Ad2xTetO-coPerth | 1.23E+07 | 3.01E+07 | 7.06E+07 | 1.87E+08 |
| AdE | 2.75E+09 | 3.80E+09 | 2.53E+09 | 4.45E+09 |

TABLE 9
Viral yield comparison between parental CAP
cells and CAP-TetR cells

| | Z3634 | | Z3635 | | C235 | |
|---|---|---|---|---|---|---|
| Viral vector | versus pCAP | | versus pCAP | | versus pCAP | |
| Ad2xTetO-tPAWHS | 64.8 | X | 200.7 | X | 345.1 | X |
| Ad2xTetO-coPerth | 2.45 | X | 5.74 | X | 15.2 | X |
| AdE | 1.38 | X | 0.92 | X | 1.62 | X |

The results of these example show that an Ad vector encoding a toxic protein can be propagated using the reagents and methods disclosed herein.

While certain embodiments have been described in terms of the preferred embodiments, it is understood that variations and modifications will occur to those skilled in the art. Therefore, it is intended that the appended claims cover all such equivalent variations that come within the scope of the following claims.

1. A recombinant polynucleotide comprising at least one Tetracycline operon repressor protein (TetR) expression cassette (TetR expression cassette), wherein each TetR expression cassette comprises: a CAG promoter/chimeric intron sequence that is optionally SEQ ID NO: 2; and, a poly(A) polynucleotide sequence that is optionally SEQ ID NO: 4 or SEQ ID NO: 6, operably linked to a coding sequence for the TetR coding sequence that is optionally SEQ ID NO: 3; optionally wherein the recombinant polynucleotide comprising a sequence having at least about 90% identity with SEQ ID NOs. 2, 3, 4 and/or 6.

2. The recombinant polynucleotide of claim 1, wherein said TetR expression cassette is SEQ ID NO.: 1 or SEQ ID NO.: 5, or an expression cassette having at least about 90% identity therewith.

3. A recombinant polynucleotide comprising at least one Tetracycline operon repressor protein (TetR) expression cassette that is optionally SEQ ID NO: 7 or an expression cassette having at least about 90% identity therewith; wherein each TetR expression cassette comprises a CMV promoter sequence that is optionally SEQ ID NO: 8 or a sequence having at least about 90% identity therewith, a rabbit beta-globin intron sequence that is optionally SEQ ID NO: 9 or a sequence having at least about 90% identity therewith, and a poly(A) polynucleotide sequence operably linked to a coding sequence for the TetR expression sequence is optionally SEQ ID NO: 3 or a sequence having at least about 90% identity therewith.

4. A TetR expressing cell comprising the recombinant polynucleotide of claim 1, integrated into the CAP cell genome.

5. The TetR expressing cell of claim 4, wherein the TetR expression cassette selected from the group consisting of SEQ ID NO: 1, SEQ ID NO: 5, SEQ ID NO: 7, and a TetR expression cassette having at least about 90% identity therewith.

6. The TetR expressing cell according to claim 4, wherein the cell is a human cell.

7. The TetR expressing cell according to claim 4, wherein the cell is an immortalized human amniocyte cell line.

8. A method for producing an adenoviral vector encoding a transgene, the method comprising:
a) obtaining a Tetracycline operon repressor protein (TetR) expressing cell comprising an exogenous polynucleotide in its genome, the polynucleotide comprising at least one Tetracycline operon repressor protein (TetR) expression cassette, each TetR expression cassette comprising a CAG promoter/chimeric intron sequence that is optionally SEQ ID NO: 2, and a poly(A) polynucleotide sequence that is optionally SEQ ID NO: 4 or SEQ ID NO: 6, operably linked to a coding sequence for the TetR gene that is optionally SEQ ID NO: 3;
b) transfecting the TetR expressing cells with at least one Tetracycline operon operator (TetO) polynucleotide sequence linked to the promoter in a recombinant Ad genomic plasmid to produce a replication-deficient Ad vector containing at least one TetO polynucleotide sequence and encoding at least one transgene; and,
c) isolating the Ad vector particles containing at least one TetO polynucleotide sequence and encoding at least one transgene;
wherein:
the polynucleotides of part a) have at least about 90% identity with any of SEQ ID NOs. 2, 3, 4 or 6; and,
the number of Ad vector particles containing at least one TetO polynucleotide sequence and encoding at least one transgene isolated in step c) is at least about any of two, 10, 15, 20, 50, 100, 150, 200, 250, or 300 times the number of Ad vector particles obtained from an Ad vector package cell lacking the TetR polynucleotide.

9. The method of claim 8, wherein the TetR expression cassette comprising SEQ ID NO: 1 or SEQ ID NO: 5, or an expression cassette having at least about 90% identity therewith.

10. The method of claim 8, wherein the chimeric intron comprises a chicken beta actin intron sequence and a rabbit beta goblin intron sequence.

11. The method of claim 8, wherein the CAG/chimeric intron sequence is according to SEQ ID NO: 2, or an intron sequence having at least about 90% identity therewith.

12. The method of claim 8, wherein the CAG/chimeric intron comprises SEQ ID NO: 2 or sequence having at least about 90% identity therewith, the poly(A) polynucleotide sequence is BGH (SEQ ID NO: 4) or a sequence having at least about 90% identity therewith, the transgene comprises a coronavirus spike protein, and the number of Ad vector particles is at least about any of 20, 60 or 200 times the number of Ad vector particles obtained from an Ad vector package cell lacking the TetR polynucleotide.

13. The method of claim 8, wherein the CAG/chimeric intron comprises SEQ ID NO: 2 or sequence having at least about 90% identity therewith, the poly(A) polynucleotide sequence is SV40 (SEQ ID NO: 6) or sequence having at least about 90% identity therewith, the transgene comprises a coronavirus spike protein, and the number of Ad vector particles is at least about any of 30, 150 or 200 times the number of Ad vector particles obtained from an Ad vector package cell lacking the TetR polynucleotide.

14. The method of claim 8, wherein the CAG/chimeric intron comprises SEQ ID NO: 2 or sequence having at least about 90% identity therewith, the poly(A) polynucleotide sequence is a BGH poly(A) polynucleotide sequence (SEQ ID NO: 4) or sequence having at least about 90% identity therewith, the transgene comprises an influenza hemagglutinin (HA) surface protein antigen from A/Perth/16/2009(H3N2), and the number of Ad vector particles is at least any of about two, 10, or 20 times the number of Ad vector particles obtained from an Ad vector package cell lacking the TetR polynucleotide.

15. The method of claim 8, wherein the CAG/chimeric intron comprises SEQ ID NO: 2 or sequence having at least about 90% identity therewith, the poly(A) polynucleotide sequence is a SV40 poly(A) polynucleotide sequence (SEQ ID NO: 6) or sequence having at least about 90% identity therewith, the transgene comprises an influenza hemagglutinin (HA) surface protein antigen from A/Perth/16/2009(H3N2), and the number of Ad vector particles is at least any of about five or 10 times the number of Ad vector particles obtained from an Ad vector package cell lacking the TetR polynucleotide.

16. A method for producing an adenoviral vector encoding a transgene, the method comprising:
a) obtaining a Tetracycline operon repressor protein (TetR) recombinant cell comprising an exogenous polynucleotide in its genome, the polynucleotide comprising at least one Tetracycline operon repressor protein (TetR) expression cassette, each TetR expression cassette comprising a CMV promoter sequence (SEQ ID NO: 8), a rabbit beta globin intron sequence (SEQ ID NO: 9), and a poly(A) polynucleotide sequence (SEQ ID NO: 4) operably linked to a coding sequence for the TetR gene (SEQ ID NO: 3);
b) transfecting the TetR expressing cells with at least one Tetracycline operon operator (TetO) polynucleotide sequence linked to the promoter in a recombinant Ad genomic plasmid to a replication-deficient Ad vector containing at least one TetO polynucleotide sequence and encoding at least one transgene; and,
c) isolating the Ad vector particles containing at least one TetO polynucleotide sequence and encoding at least one transgene;
wherein:
the polynucleotides of part a) have at least about 90% identity with any of SEQ ID NOs. 3, 4 8, or 9; and,
the number of Ad vector particles containing at least one TetO polynucleotide sequence and encoding at least one transgene isolated in step c) is at least about any of two, 10, 15, 20, 50, 100, 150, 200, 250, or 300 times the number of Ad vector particles obtained from an Ad vector package cell lacking the TetR polynucleotide.

17. The method of claim 16, wherein the TetR expression cassette is according to SEQ ID NO: 7 or an expression cassette have at least about 90% identity therewith.

18. The method of claim 14, wherein the intron sequence is SEQ ID NO: 9 or an intron sequence having at least about 90% identity therewith.

19. The method of claim 8, wherein the poly(A) polynucleotide sequence is a BGH or SV40 poly(A) polynucleotide sequence, optionally SEQ ID NO: 4 or SEQ ID NO: 6 or a polynucleotide sequence having at least about 90% identity therewith.

20. The method of claim 8, wherein the transgene is a viral or bacterial antigen.

21. The method of claim 20, wherein the viral antigen is a coronavirus or flu antigen.

22. The method of claim 16, wherein the rabbit beta globin intron sequence comprises SEQ ID NO: 9 or a sequence having at least about 90% identity therewith, the poly(A) polynucleotide sequence is a BGH poly(A) polynucleotide sequence (SEQ ID NO: 4) or a polynucleotide sequence having at least about 90% identity therewith, the transgene comprises a coronavirus spike protein, and the number of Ad vector particles is at least about any of 30, 85 or 350 times the number of Ad vector particles obtained from an Ad vector package cell lacking the TetR polynucleotide.

23. The method of claim 16, wherein the rabbit beta globin intron sequence comprises SEQ ID NO: 9 or a sequence having at least about 90% identity therewith, the poly(A) polynucleotide sequence is a BGH poly(A) polynucleotide sequence (SEQ ID NO: 4) or a sequence having at least about 90% identity therewith, the transgene comprises an influenza hemagglutinin (HA) surface protein antigen from A/Perth/16/2009(H3N2), and the number of Ad vector particles is at least any of about 5 or 15 times the number of Ad vector particles obtained from an Ad vector package cell lacking the TetR polynucleotide.

24. An adenoviral particle prepared by claim 8.
